(19) United States
(12) Patent Application Publication
(10) Pub. No.: US 2006/0165732 A1
(43) Pub. Date: Jul. 27, 2006

(54) Fungal antigens and eosinophil activity

(76) Inventors: Jens Ponikau, Rochester, MN (US); Hirohito Kita, Rochester, MN (US); David A. Sherris, Buffalo, NY (US)

Correspondence Address: FISH & RICHARDSON P.C., PO BOX 1022, MINNEAPOLIS, MN, 55440-1022, US

(21) Appl. No.: 10/505,379

(22) PCT Filed: Feb. 28, 2003
(86) PCT No.: PCT/US03/06380

§ 371 Date: Jul. 27, 2005

(30) Foreign Application Priority Data
Mar. 1, 2002 (US) 60361211
Feb. 13, 2003 (US) 60447840

(51) Int. Cl. A61K 39/00; A61K 36/06; C12Q 1/18

(52) U.S. Cl. 424/274.1; 424/195.16; 435/32

(57) ABSTRACT

The invention provides methods and materials related to T cell activation, eosinophil activation, and the ability of eosinophils to attack fungi. The invention also provides animals having a fungal antigen-induced eosinophilic response as well as methods of making such animals and method of using such animals to identify compounds that inhibit an eosinophilic response in an animal.

### BACKGROUND

1. Technical Field

The invention relates to methods and materials involved in eosinophil activation such as methods for identifying activators and inhibitors of T-cell activation, eosinophil activation, and fungus attack.

2. Background Information

Mucositis, the inflammation of mucosal tissue, is a serious medical problem that affects millions of people. Conservative estimates indicate that between 20 to 40 million Americans suffer from chronic rhinosinusitis (CRS), for example, an inflammation of the nasal cavity and/or paranasal sinuses.

For the most part, the cause of CRS is unknown. In some patients, however, non-invasive fungal organisms living within the mucus seem to be involved. Patients having this condition, referred to as allergic fungal sinusitis (AFS), were first described in the early 1980's (Miller et al. (1981) Prod. Scot. Thor. Soc. 36:710; and Katzenstein et al. (1983) J. Allergy Clin. Immunol. 72:89-93). Briefly, AFS is diagnosed by the presence of inspissated mucus in the nasal-paranasal cavities. This mucus typically contains clumps or sheets of necrotic eosinophils, Charcot-Leyden crystals, and non-invasive fungal hyphae. In addition, patients with AFS often have a history of nasal-paranasal polyposis and may have undergone multiple surgeries. Inflammation can affect all nasal-paranasal cavities, but also can be asymmetric, and only involve one side. Computed topography (CT) scans of patients with AFS have a characteristic appearance and often reveal bone erosion in adjacent structures. Indeed, destruction of bones adjacent to the sinuses and nasal areas ranging from 19 percent to 80 percent has been reported.

Individuals suffering from CRS typically experience repeated cycles of intense inflammation, surgery, and steroid therapy followed by recurrent intense inflammation. Neither surgery nor steroid therapy, however, is particularly effective or desirable as a long-term treatment for CRS. A method for controlling the inflammatory response seen in CRS patients would be particularly useful for treating the condition.

### SUMMARY

The invention provides methods and materials related to T cell activation, eosinophil activation, and the ability of eosinophils to attack fungi. For example, the invention provides methods and materials for identifying compounds that inhibit fungus-induced T cell activation as well as methods and materials for identifying fungal antigens capable of inducing T cell activation. The invention also provides methods and materials for identifying compounds that inhibit fungus-induced eosinophil activation and degranulation as well as methods and materials for identifying fungal antigens capable of inducing eosinophil activation and degranulation. In addition, the invention provides methods and materials for identifying compounds that inhibit the ability of eosinophils to attack fungi as well as methods and materials for identifying fungal antigens capable of triggering eosinophils to attack fungi. The methods and materials provided herein can be used to develop successful treatments for fungus-induced mucositis conditions.

The invention also provides animals having a fungal antigen-induced eosinophilic response as well as methods of making such animals and method of using such animals to identify compounds that inhibit an eosinophilic response in an animal.

In one aspect, the invention features a method of identifying an inhibitor of fungus-induced eosinophil degranulation. The method includes determining whether or not a test compound reduces the amount of eosinophil degranulation induced by a fungal preparation, wherein such reduction indicates that the test compound is an inhibitor. The fungal preparation can be a cellular fungus extract (e.g., an Alternaria extract, a Candida extract, an Aspergillus extract, or a Cladisporium extract). Alternatively, the fungal preparation can be a supernatant collected from a fungus culture (e.g., an Alternaria culture, a Candida culture, an Aspergillus culture, or a Cladisporium culture). The amount of eosinophil degranulation can be determined by measuring major basic protein (MBP) or eosinophil-derived neurotoxin (EDN).

In another aspect, the invention features a method of identifying a fungal component that induces eosinophil degranulation. The method includes contacting an eosinophil with a test component from a fungus, and determining whether or not the test component induces the eosinophil to degranulate, wherein the presence of degranulation indicates that the test component is a fungal component that induces eosinophil degranulation. The test component can be a polypeptide obtained from a fungus extract (e.g., an Alternaria extract, a Candida extract, an Aspergillus extract, or a Cladisporium extract), or obtained from a fraction of such a fungus extract. The test component can be a polypeptide obtained from the supernatant of a fungus culture (e.g., an Alternaria culture, a Candida culture, an Aspergillus culture, or a Cladisporium culture), or obtained from a fraction of such a supernatant. The level of eosinophil degranulation can be determined by measuring MBP or EDN.

In another aspect, the invention features a method of identifying an inhibitor of eosinophil fungus attack. The method includes determining whether or not a test compound reduces the amount of eosinophil fungus attack induced by a sample (e.g., a supernatant) obtained from a culture of cells from a chronic rhinosinusitis patient and a fungal preparation, wherein reduction of eosinophil attack indicates that the test compound is an inhibitor. The cells can be peripheral blood mononuclear cells. The fungal preparation can be a cellular fungus extract (e.g., an Alternaria extract, a Candida extract, an Aspergillus extract, or a Cladisporium extract). Alternatively, the fungal preparation can be media collected from a fungus culture (e.g., an Alternaria culture, a Candida culture, an Aspergillus culture, or a Cladisporium culture). The amount of eosinophil fungus attack can be determined by light microscopy.

In another aspect, the invention features a method of identifying a factor that induces eosinophil fungus attack. The method includes contacting an eosinophil with a test component from a sample (e.g., a supernatant) obtained from a culture containing cells from a chronic rhinosinusitis patient and a fungal preparation, and determining whether or not the test component induces the eosinophil to attack fungus, wherein the presence of eosinophil fungus attack indicates that the test component is a factor that induces eosinophil fungus attack. The test component can be a polypeptide produced by a T cell from a chronic rhinosinusitis patient. The cells can be peripheral blood mononuclear cells. The fungal preparation can be a cellular fungus extract (e.g., an Alternaria extract, a Candida extract, an Aspergillus extract, or a Cladisporium extract). Alternatively, the fungal preparation can be media collected from a fungus culture (e.g., an Alternaria culture, a Candida culture, an Aspergillus culture, or a Cladisporium culture). The amount of eosinophil fungus attack can be determined by light microscopy.

In yet another aspect, the invention features a method of identifying an inhibitor of a T cell response to fungus. The method includes determining whether or not a test compound reduces the amount of activation of T cells induced by a sample comprising a fungal preparation, wherein the T cells are from a chronic rhinosinusitis patient, and a reduction in the amount of T cell activation indicates that the test compound is an inhibitor. The sample can contain peripheral blood mononuclear cells from the chronic rhinosinusitis patient. The fungal preparation can be a cellular fungus extract (e.g., an Alternaria extract, a Candida extract, an Aspergillus extract, or a Cladisporium extract). Alternatively, the fungal preparation can be media collected from a fungus culture (e.g., an Alternaria culture, a Candida culture, an Aspergillus culture, or a Cladisporium culture). The amount of activation of the T cells can be determined by measuring interleukin-5, interleukine-13, or interferon-γ.

In another aspect, the invention features a method of identifying a fungal antigen that induces a T cell response to fungus in a patient having chronic rhinosinusitis. The method includes incubating a T cell with antigen presenting cells and a test antigen, wherein the T cell is from a chronic rhinosinusitis patient, and the test antigen is a molecule of a fungus, and determining whether or not the test antigen induces activation of the T cell. The presence of activation of the T cell indicates that the test antigen is the fungal antigen. The antigen presenting cells can be contained within peripheral blood mononuclear cells from the chronic rhinosinusitis patient. The fungus can be selected from the group consisting of Alternaria, Candida, Aspergillus, and Cladisporium. The amount of activation of the T cells can be determined by measuring interleukin-5, interleukin-13, or interferon-γ.

In another aspect, the invention features a method of screening for a compound that inhibits an eosinophilic response. The method includes contacting an animal with a fungal antigen in the presence of a test compound and comparing the amount of eosinophils in the animal with the amount of eosinophils in a control animal. The control animal is an animal that was contacted with the fungal antigen in the absence of the test compound. Typically, a decrease in the amount of eosinophils in the animal relative to the control animal indicates that the test compound inhibits the eosinophilic response.

In another aspect, the invention features a mouse comprising an eosinophilic response induced by administration of a fungal antigen to the nasal passages of the mouse. Typically, the eosinophilic response is present within a portion of the lungs of the mouse. The fungal antigen can be an Alternaria antigen.

In another aspect, the invention features a method for identifying a compound that inhibits an eosinophilic response. The method includes: (a) contacting an animal with a fungal antigen to induce eosinophilia in the animal, (b) administering a test compound to the animal, and (c) determining whether or not the test compound reduced the eosinophilia, wherein a reduction in the eosinophilia indicates that the test compound is a compound that inhibits an eosinophilic response. The fungal antigen can be an Alternaria antigen. The animal can be a mouse. The eosinophilia can be present in the lungs of the animal.

In another aspect, the invention features a method for identifying a compound that inhibits an eosinophilic response. The method includes: (a) contacting an animal with a fungal antigen to induce eosinophilia in the animal, (b) administering a test compound to the animal, and (c) comparing the amount of eosinophilia in the animal with the amount of eosinophilia in a control animal contacted with the fungal antigen and not the test compound, wherein a decrease in the amount of eosinophilia in the animal relative to the control animal indicates that the test compound is a compound that inhibits an eosinophilic response. The fungal antigen can be an Alternaria antigen. The animal can be a mouse. The eosinophilia can be present in the lungs of the animal.

Another aspect of the invention features a non-human animal (e.g., mouse) containing an eosinophilic response induced by administration of a fungal antigen to the nasal passages of the animal, wherein the eosinophilic response is present within a portion of the lungs of the animal. The fungal antigen can be an Alternaria antigen.

Another aspect of the invention features a population of non-human animals, wherein the population contains more than 3 animals, wherein each animal of the population contains an eosinophilic response induced by administration of a fungal antigen to the nasal passages of each animal, and wherein the eosinophilic response is present within a portion of the lungs of each animal. The fungal antigen can be an Alternaria antigen. Each animal can be a mouse. The population can contain more than 5 animals or more than 10 animals.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a bar graph showing the effects of Alternaria and Candida culture supernatants and cellular extracts on IL-5 release from the PBMC of normal subjects and CRS patients.

FIG. 2 is a bar graph showing the effects of Alternaria and Candida culture supernatants and cellular extracts on IL 13 release from the PBMC of normal subjects and CRS patients.

FIG. 3A is a bar graph showing the effects of 50 μg/mL cellular extracts from Alternaria, Aspergillus, Candida, Cladisporium, Penicillium, and control medium on eosinophil degranulation, as measured by EDN release. Data are presented as mean±SEM from six different eosinophil preparations. The asterisk denotes significant differences compared with medium alone (p<0.05).

FIG. 3B is a bar graph plotting the amount of EDN release from eosinophils isolated from non-allergic (n=11) and allergic (n=10) donors and treated with an Alternaria extract or IL-5. Data are presented as mean±SEM. The asterisk denotes a significant difference between the cells from allergic and non-allergic donors (p<0.05).

FIG. 3C is a bar graph plotting the amount of EDN release from eosinophils receiving the indicated treatment (n=9). Data are presented as mean±SEM.

FIG. 3D is a line graph plotting the time course of EDN release induced by media, an Alternaria cellular extract (100 μg/mL), extract from an Alternaria culture, or IL-5 (10 ng/mL). Data are presented as mean±SEM from three different eosinophil preparations.

FIG. 4 is a light micrograph showing the clustering of eosinophils around fungal hyphae after 1 hour (left panel) and 15 hours (right panel) of incubation, following treatment of the eosinophils with supernatants from CRS patient PBMC incubated with fungal antigens.

FIG. 5 is a light micrograph showing that untreated eosinophils do not cluster around fungal hyphae, even after 15 hours.

FIG. 6A is a bar graph plotting the amount of EDN release from eosinophils receiving the indicated treatment. Eosinophils were incubated in duplicate with non-treated (NT) or heat-treated 100 μg/mL Alternaria cellular (Cell) extract or Alternaria culture extract (Cult). The heat treatment was a 10 minute incubation as 100° C. Data are presented as mean±SEM from five different eosinophil preparations. The asterisk denotes a significant difference between the heat-treated and non-treated experiments (p<0.05).

FIG. 6B is a bar graph plotting the amount of EDN release from eosinophils receiving the indicated treatment at either 37° C. or 4° C. Data are presented as mean±SEM from five different eosinophil preparations. The asterisk denotes a significant difference between the 37° C. and 4° C. incubations for a particular treatment p<0.05).

FIG. 7 contains two bar graphs plotting the amount of EDN released from eosinophils receiving the indicated treatment plus 5 μg/mL Cytochalasin B (FIG. 7A) or 10 μg/mL anti-CD18 mAb (FIG. 7B). Controls were cells receiving the indicated treatment alone (non-treatment; NT) or in combination with 10 μg/mL mouse IgG1. The Alternaria treatment was with 100 rig/mL Alternaria culture extract, while the IL-5 treatment was with 10 ng/mL IL-5. Data are presented as mean±SEM from four (FIG. 7A) or six (FIG. 7B) different eosinophil preparations. The asterisk denotes a significant difference when compared to NT result within a particular group (p<0.05).

FIG. 8 contains two bar graphs plotting the amount of EDN released from eosinophils receiving the indicated treatment plus extracellular calcium (FIG. 8A) or EGTA (FIG. 8B). Eosinophils were preincubated with different concentration of calcium (FIG. 8A) or EGTA (FIG. 8B) and stimulated with medium alone, 100 μg/mL Alternaria culture extract, or 10 ng/mL IL-5. Data are presented as mean±SEM from four (FIG. 8A) or five (FIG. 8B) different eosinophil preparations. The asterisk denotes a significant difference when compared to the 1.2 mM calcium treatment (FIG. 8A) or the 0 mM EGTA treatment (FIG. 8B) (p<0.05).

FIG. 8C is a graph plotting the changes in [Ca²⁺]i in eosinophils stimulated with medium, Alternaria culture extract, or IL-5. Eosinophils were pretreated with the calcium-sensitive fluorescent dye indo-1/AM, loaded onto the FACS analyzer, and stimulated after 60 seconds with medium, 100 μg/mL Alternaria culture extract, or 10 ng/mL IL-5. [Ca²⁺]i is shown as the ratio of the calcium-bound indo-1 fluorescence emission (401 nm) to the free indo-1 emission (475 nM). Arrow indicates the time of addition of medium, Alternaria culture extract, or IL-5.

FIGS. 8D and 8E are bar graphs plotting the amount of EDN released from eosinophils receiving the indicated treatment plus ionomycin (FIG. 8D) or thapsigargin (FIG. 8E). Eosinophils were preincubated with different concentration of ionomycin (FIG. 8D) or thapsigargin (FIG. 8E), and stimulated with medium, 100 μg/mL Alternaria culture supernatant extract, or 10 ng/mL IL-5. Data are presented as mean±SEM from six different eosinophil preparations. The asterisk denotes a significant difference when compared to the 0 nM treatment of each group p<0.05).

FIG. 9 is a bar graph plotting the amount of IL-8 produced by eosinophils receiving the indicated treatment. Eosinophils were incubated with 50 or 100 μg/mL Alternaria extract, or 10 ng/mL IL-5. Data are presented as mean±SEM from three different eosinophil preparations. The asterisk denotes a significant difference when compared to the result obtained with medium only (p<0.05).

FIG. 10A is a bar graph showing the immune cells in BAL fluids following sensitization and challenge of mice with Alternaria extracts or OVA.

FIG. 10B is a bar graph showing the immune cells in BAL fluids following challenge of unsensitized mice with fungal cellular or culture extracts, or with OVA.

FIG. 11 is a line graph showing the airway reactivity of mice challenged with fungal cellular or culture extracts, or with OVA.

FIG. 12 is a bar graph showing the effects of anti-IL-5 antibodies on Alternaria-induced eosinophilia in the airway of a mouse.

FIG. 13 contains four bar graphs showing the level of IL-5 (FIG. 13A), IL-4 (FIG. 13B), IFN-γ (FIG. 13C), and IL-13 (FIG. 13D) in BAL fluids of mice challenged with Alternaria extracts. The arrows indicate the time points when the mice were challenged with Alternaria extracts.

FIG. 14A contains two bar graphs showing the amount of cytokine production by naïve beige mice and naïve Rag1 knockout mice 12 hours after challenge with Alternaria. The amount of IL-5 and IFN produced by challenged Beige mice is shown in the left panel, and the amount of IL-5 produced by challenged Rag1 knockout mice is shown in the right panel.

FIG. 14B contains two bar graphs showing the amount of cytoline production by naïve beige mice and naïve Rag1 knockout mice 8 days after challenge with Alternaria. The amount of IL-5 produced by Beige mice is shown in the left panel, and the amount of IL-5 and IFN produced by Rag1 knockout mice is shown in the right panel.

FIG. 14C contains two bar graphs showing the immune cells present in Alternaria-induced airway inflammation. The types of immune cells produced in challenged Beige mice is shown in the left panel, and the types of immune cells produced in challenged Rag1 knockout mice is shown in the right panel.

FIG. 15 contains two line graphs showing IL-5 production in lung tissue exposed to Alternaria in vitro. The line graph of FIG. 15A plots IL-5 production in lung tissue over time, while the line graph of FIG. 15B plots IL-5 production in response to varying doses of Alternaria extract.

### DETAILED DESCRIPTION

The invention provides methods and materials for identifying activators and inhibitors of eosinophil degranulation, eosinophil attack of fingi, and T cell activation. These methods and materials can be used to identifying compounds for treating the inflammation that results from mucositis, such as that seen in CRS patients. Without being bound by a particular mechanism, the inflammation that occurs with CRS appears to be the result of a process that is initiated by the presentation of one or more antigens from a fungal cell located in the mucus of a mammal (e.g., nasal or sinus mucus). The presentation of the one or more antigens by an antigen presenting cell (APC) may stimulate T cells to release signaling molecules such as cytokines, which play a role in the recruitment and activation of eosinophils. Activated eosinophils can undergo degranulation as described below, thus releasing numerous toxins into the surrounding milieu. If the activated eosinophils migrate from tissues (e.g., nasal tissues) into the mucus (e.g., nasal mucus), then they can “attack” the fungal cells by localizing to and degranulating on the cells, thus leading to fungal cell death. A fungal antigen, possibly a protease product, also appears to stimulate eosinophil degranulation directly, however, without activation through the T cell pathway.

CRS and Non-Invasive, Fungus-Induced Mucositis

The term “chronic” as used herein refers to afflictions present for at least three months. It is to be understood that afflictions that are treated and subsequently become asymptomatic can be classified as chronic. Thus, chronic afflictions can be symptomatic or asymptomatic.

The term “mucositis” as used herein means an inflammation, as opposed to an infection, of a mucus membrane. A “non-invasive fungus-induced” mucositis therefore is an inflammation of a mucus membrane caused by a fungus that does not enter the tissue. Fungus-induced CRS is an example of a non-invasive fungus-induced mucositis.

In general, inflammation is fundamentally and clinically different from infection. An infection is defined as the growth of an organism within tissue. In addition, an infection is characterized as an invasive disease, meaning that an infectious organism enters the tissue of a host and then triggers a host immune response and/or causes damage. In contrast, an inflammation is characterized as a localized protective response that serves to destroy, dilute, and/or sequester an injurious agent or insult. In addition, inflammatory responses typically result in redness, swelling, heat, and pain. In the case of non-invasive fungus-induced mucositis, the localized protective response is against a non-invasive fungal organism living outside the tissue (e.g., within mucus). Typically, some individuals suffering from a non-invasive fungus-induced mucositis are atopic and/or immunocompetent. In addition, the role of the injurious agent (i.e., fungus) is that of a non-invasive allergen. Thus, a non-invasive fungus-induced mucositis is an allergic reaction mounted by an individual's immune system against a fungal organism living outside the individual's tissue.

Most, if not all, individuals have fungal organisms living in their mucus. Most individuals normally tolerate these non-invasive organisms and live normal disease-free lives. For unknown reasons, however, some individuals do not tolerate these fungal organisms and begin to mount an immune response against them. As the immune response progresses, eosinophils accumulate within the local tissue. This accumulation of eosinophils can contribute to the formation of obstructive tissue masses (e.g., polyps and polypoid structures) as well as the transmigration of activated eosinophils from the tissue (inside the body) to the mucus (outside the body). These obstructive tissue masses appear to prevent normal cavity clearance and thus can facilitate additional fungal growth.

Once eosinophils are within the mucus, they can degranulate, releasing the toxic contents of their granules. Upon release, these toxic molecules can damage both the targeted foreign microorganisms (e.g., fungus) as well as self tissues. The degree of damage caused by eosinophil accumulation and degranulation varies significantly, ranging from slight inflammatory pain and discomfort to major structural abnormalities such as tissue and bone destruction and the formation of polyps, polypoid structures, and other tumors. Damage to self tissues can lead to increased susceptibility to bacterial infections as well. Thus, the characteristic inflammatory responses, subsequent damage, and resulting bacterial infections observed in most CRS patients actually are triggered by non-invasive fungal organisms.

Any fungal organism living in the mucus of a mammal can be a non-invasive fungal organism that is capable of inducing mucositis, since it is the presence of the organism in an intolerant individual's mucus that causes inflammation. For example, all fungal organisms previously identified in mucus samples of AFS patients can be non-invasive fungal organisms capable of inducing non-invasive fungus-induced mucositis. These include, without limitation, Absidia, Aspergillus flavus, Aspergillus fumigatus, Aspergillus glaucus, Aspergillus nidulans, Aspergillus versicolor, Alternaria, Basidiobolus, Bipolaris, Candida albicans, Candida lypolytica, Candida parapsilosis, Cladosporium, Conidiobolus, Cunninahamella, Curvularia, Dreschlera, Exserohilum, Fusarium, Malbranchia, Paecilomvces, Penicillium, Pseudallescheria, Rhizopus, Schizophylum, and Sporothrix. Other examples of fungal organisms that may be capable of causing a non-invasive fungus-induced mucositis include, without limitation, Acremonium, Arachniotus citrinus, Aurobasidioum, Beauveria, Chaetomium, Chryosporium, Epicoccum, Exophilia jeanselmei, Geotrichum, Oidiodendron, Phoma, Pithomyces, Rhinocladiella, Rhodoturula, Sagrahamala, Scolebasidium, Scopulariopsis, Ustilago, Trichoderma, and Zygomycete. A list of additional fungal organisms that can be capable of inducing a non-invasive fungus-induced mucositis can be found in most taxonomic mycology textbooks.

Fungus-Induced Eosinophil Degranulation

Eosinophils belong to the granulocyte class of white blood cells, and contain cytoplasmic granules that stain with the acidic dye eosin. Eosinophils are the main effectors of antibody-dependent cell-mediated cytotoxicity against multicellular parasites that provoke IgE antibodies. Their role seems to be to engulf and destroy the precipitated antigen-antibody complexes produced in humorally based immune reactions. An elevated eosinophil count usually is seen in allergic reactions, and numerous eosinophils are chemotactically aggregated at sites where antigen-antibody complexes are found.

As used herein, “fungus-induced eosinophil degranulation” refers to eosinophil degranulation in response to one or more antigens from fungal cells (e.g., from fungal cell extracts or fungal culture supernatants). Degranulation is the release of toxic molecules such as eosinophil cationic protein (ECP), eosinophil peroxidase (EPO), and MBP that are contained within eosinophil granules; this release typically causes damage to or death of cells in the vicinity of the degranulating eosinophils.

Eosinophil degranulation can be achieved in vitro as described in Example 2, for example. By this method, a fungal preparation (e.g., a fungal cell extract or fungal culture supernatant) can be added to an eosinophil to induce degranulation. As used herein, a “fungal cell extract” is a preparation that contains factors (e.g., polypeptides) found within a fungal cell (e.g., in the cytoplasm, membranes, or organelles of a fungal cell). The term “fungal culture supernatant” refers to media obtained from culturing fungal cells. A fungal culture supernatant can be manipulated to form solid material. For example, a fungal culture supernatant can be obtained by removing fungal organisms from a fungal culture. The resulting supernatant then can be concentrated such that any remaining material (e.g., fungal polypeptides) form concentrated liquid or dry material. This dry material can be a fungal culture extract.

A cell extract or culture supernatant from any suitable type of fungus can be used to induce degranulation, including extracts and supernatants from those fungi listed above (e.g., Alternaria, Candida, Aspergillus, or Cladisporium). Alternaria cell extracts and culture supernatants are particularly useful. These can be obtained by standard laboratory cell culture and extract preparation techniques. Alternatively, fungal cell extracts and culture supernatants are commercially available (e.g., from Greer Laboratories, Lenoir, N.C.). Eosinophils can be obtained by, for example, purification from an individual's blood. Methods for such purification are known in the art.

Eosinophil degranulation can be stimulated in vitro by, for example, incubating a fungal preparation (e.g., a volume of Alternaria culture supernatant or 50 μg/mL of an Alternaria culture supernatant extract) with an eosinophil (e.g., purified eosinophils). Any incubation time (e.g., 1, 2, 3, 4, 5, 6, 7, or more hours) can be used. For example, an incubation time from about 2 to about 6 hours can be used. Any amount of a fungal preparation can be used. For example, the amount of a fungal extract can range from about 10 g/mL to about 100 mg/mL (e.g., about 50, 100, 200, 300, or more μg/mL). Degranulation can be measured by a number of methods, including those known in the art. Degranulation can be assessed by, for example, measuring the release of markers such as ECP, EPO, MBP, or EDN. Non-limiting examples of methods for measuring marker levels include protein-based methods such as ELISA assays and western blotting. Alternatively, degranulation can be assessed by visual inspection of eosinophils by microscopy (e.g., using an electron microscope) to detect the presence of empty granules.

Identifying an Inhibitor of Fungus-Induced Eosinophil Degranulation

The invention provides methods and materials that can be used to identify a compound that inhibits fungus-induced eosinophil degranulation. For example, an inhibitor of fungus-induced eosinophil degranulation can be identified by contacting an eosinophil with a fungal preparation (e.g., an Alternaria culture supernatant, a Candida extract, or an Aspergillus culture supernatant) in the presence and absence of a test compound, and measuring levels of degranulation (e.g., by measuring EDN output or MBP output, or by observing empty granules within eosinophils viewed by microscopy). A test compound can be identified as an inhibitor of eosinophil degranulation if the level of degranulation is reduced in the presence of the compound as compared to the level of degranulation observed in the absence of the test compound. By “reduced” is meant that the level of degranulation in the presence of the test compound is less (e.g., 1% less, 5% less, 10% less, 50% less, 90% less, or 100% less) than the level observed without the test compound.

Molecules belonging to any of a number of classes can be used as test compounds. For example, molecules that are polypeptides (i.e., amino acid chains of any length, regardless of modification such as phosphorylation or glycosylation), oligonucleotides, esters, lipids, carbohydrates, and steroids can be used as test compounds. Molecules that are protease inhibitors may be particularly useful, as described in Example 3. Such protease inhibitors can be included within a cocktail of inhibitors (e.g., inhibitor cocktails that are commercially available from Roche Molecular Biochemicals, Indianapolis, Ind.) or can be individual protease inhibitors (e.g., a single serine protease inhibitor such as AEBSF).

Identifying Degranulation Inducing Molecules

The invention provides methods and materials that can be used to identify a fungal component that stimulates eosinophil degranulation. A degranulation inducing fungal component can be, for example, a fungal antigen. By way of example and not limitation, a method for identifying a fungal component that induces eosinophil degranulation can involve contacting an eosinophil with a test component and determining whether degranulation occurs in the presence of the test component. A test component “stimulates eosinophil degranulation” if the level of eosinophil degranulation is observed to be higher (e.g., 1% higher, 5% higher, 10% higher, 50% higher, 100% higher, or more) in the presence of the component than the level of degranulation observed in the absence of the component. As described above, a number of methods can be used to assess the level of degranulation (e.g., measuring EDN or MBP output, or observing empty granules within eosinophils viewed under a microscope).

Eosinophils can be obtained from the blood of a normal subject or from a CRS patient, for example. A suitable test component typically is a fungal preparation (e.g., a fungal cell extract or a fungal culture supernatant, a fraction of such extracts or supernatants, or a purified fungal molecule). These can be obtained from any suitable fungus (e.g., Alternaria, Candida, Aspergillus, or Cladisporium) or can be synthetically made. For example, a fungal polypeptide fragment can be synthesized using a polypeptide synthesizer. Purified Alternaria polypeptides may be particularly useful. The fractionating of extracts or supernatants refers to the process of separating molecules within an extract or a supernatant into separate portions based on characteristics such as, for example, molecular size or charge. Non-limiting examples of methods for fractionating cellular extracts or culture supernatants include column chromatography (e.g., using a gel exclusion or an ion exchange column), and gel electrophoresis followed by excision and purification of a particular section of the gel.

Eosinophil Fungus Attack

As used herein, “eosinophil attack” refers to the localization of an eosinophil to a particular cell or organism, such that the eosinophil can release its toxins directly to the surface of the selected cell or organism. Such localization typically involves direct contact between an eosinophil and a target cell. An “eosinophil fungus attack” refers to the localization of an eosinophil to the surface of a fungal cell. A “fungus-induced eosinophil attack,” as used herein, is an eosinophil attack that is stimulated by a fungal factor.

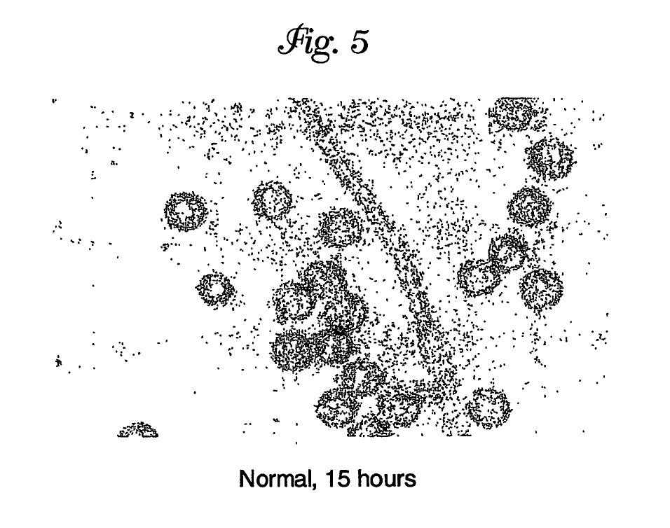

As described below (see Example 4), eosinophil attack of fungal cells can be stimulated by contacting eosinophils with supernatants obtained from culturing cells from a CRS patient with a fungal preparation (e.g., a fungal cell extract or a fungal culture supernatant) in the presence of an APC. Eosinophils that are stimulated with such supernatants can localize to fungal cells and undergo degranulation, thus killing the fungal cells. As described in Example 4 and shown in FIG. 5, fungus-induced eosinophil attack typically does not occur when the supernatant is obtained by incubating T cells from normal subjects with fungal antigens and APC.

Methods of inducing eosinophil attack can utilize T cells obtained by, for example, isolating T cells from the blood of a CRS patient, or by obtaining peripheral blood mononuclear cells (PBMC) from a CRS patient. Immortalized T cell lines also can be used, especially if such lines are isolated from a CRS patient. Fungal preparations can be from any suitable species, including those listed above. Extracts and supernatants from Alternaria, Candida, Aspergillus, and Cladisporium, for example, can be used. Such extracts and supernatants can be obtained using standard laboratory protocols, and also are commercially available. APC can be those contained within a T cell-containing PBMC isolate obtained from a CRS patient, for example. Alternatively, purified APC (e.g., dendritic cells, macrophages, or B cells) can be used, and can be isolated from a normal subject or a CRS patient or commercially obtained.

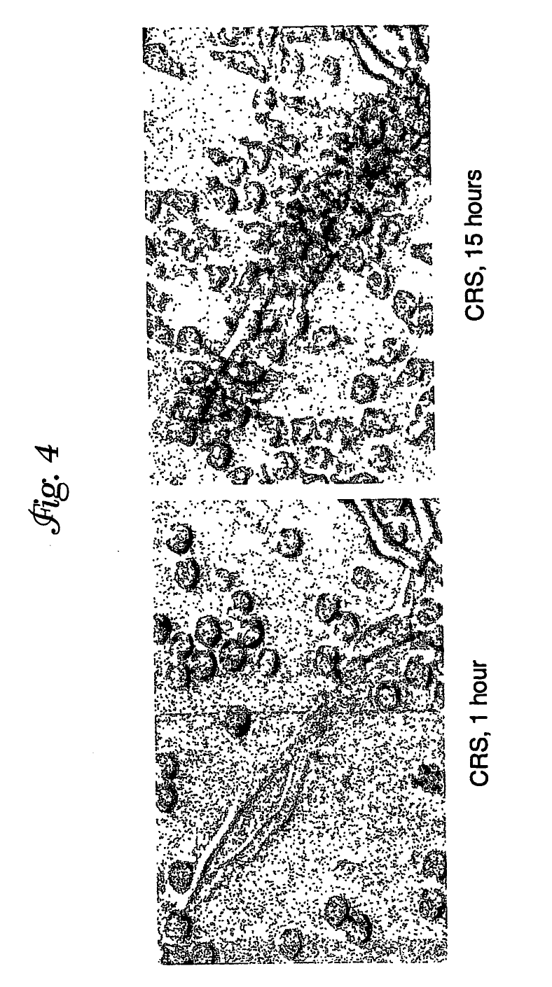

Eosinophil attack can be observed by, for example, microscopy (e.g., using a light microscope; see FIGS. 4 and 5). Such methods permit visualization of eosinophil localization to a fungal cell, as well as the degranulation of the eosinophil. Alternatively, eosinophil attack can be detected by other methods including, for example, panning (e.g., detection of the interaction between a labeled eosinophil and a fungal cell fixed to a support), and coimmunoprecipitation using an antibody specific for an eosinophil surface protein followed by, for example, fungal cell staining or visualization by microscopy.

Identifying an Inhibitor of Eosinophil Fungus Attack

The invention provides methods and materials that can be used to identify a compound that inhibits eosinophil attack. As described above, eosinophil attack can be detected by, for example, visually assessing whether eosinophils localize to target cells (e.g., fungal cells).

A method of identifying an inhibitor of eosinophil attack can involve incubating a cell from a CRS patient (e.g., a T cell) with a fungal preparation and an APC in the presence and absence of a test compound, collecting the supernatant from the incubation, adding the supernatant to an eosinophil in the presence of a fungal cell (e.g., an Alternaria cell, a Candida cell, an Aspergillus cell, or a Cladisporium cell), and observing whether the eosinophil attacks the fungal cell (e.g., by using microscopy to detect eosinophil localization to the fungal cell). A test compound can be identified as an inhibitor of eosinophil fungus attack if eosinophil localization to the fungal cell is reduced in the presence of the compound as compared to in the absence of the compound. By “reduced” is meant that the occurrence of eosinophil attack of a fungal cell is lower (e.g., 1%, 5%, 10%, 50%, or 100% lower) in the presence of the test compound than in the absence of the compound.

A method of identifying an inhibitor of eosinophil attack can involve incubating eosinophils with (1) a sample obtained from culturing cells from a CRS patient (e.g., PBMC or T cells plus APC) with a fungal preparation, and (2) a fungal cell (e.g., an Alternaria cell, a Candida cell, an Aspergillus cell, or a Cladisporium cell), in the presence and absence of a test compound. During or after this incubation, the mixture can be assessed to determine whether the eosinophils attacked the fungal cell (e.g., by using microscopy to detect eosinophil localization to the fungal cell). A test compound can be identified as an inhibitor of eosinophil fungus attack if eosinophil localization to the fungal cell is reduced in the presence of the compound as compared to in the absence of the compound. By “reduced” is meant that the occurrence of eosinophil attack of a fungal cell is lower (e.g., 1%, 5%, 10%, 50%, or 100% lower) in the presence of the test compound than in the absence of the compound.

Eosinophils can be obtained from the blood of a CRS patient or a normal subject, for example. T cells also can be purified from the blood of a CRS patient, or can be contained within a PBMC preparation from a CRS patient. T cells from an immortalized cell line can be used, especially if the line was originated in a CRS patient. APC can be, for example, contained within the same CRS patient PBMC isolate that can serve as the source of T cells. Alternatively, purified APC (e.g., dendritic cells, macrophages, or B cells) also can be used. Useful fungal preparations include, for example, cell extracts and fungal culture supernatants from organisms such as those listed above (e.g., Alternaria, Candida, Aspergillus, and Cladisporium). Suitable test compounds can belong to any of a number of classes. For example, molecules that are polypeptides, oligonucleotides, esters, lipids, esters, carbohydrates, or steroids can be used as test compounds. Those of ordinary skill in the art can readily establish suitable amounts of test compounds and suitable incubation times.

Identifying an Activator of Eosinophil Fungus Attack

The invention provides methods and materials that can be used to identify a factor that is capable of inducing eosinophil attack. Eosinophil attack can be assessed by, for example, observing whether eosinophils localize to target cells (e.g., fungal cells) in response to treatment with a particular compound or factor.

An activator of eosinophil attack can be, for example, a factor that is released by the T cells of a CRS patient but not by the T cells of a normal subject following incubation with a particular fungal antigen in the presence of an APC. Such an inducer of eosinophil attack can be identified by, for example, incubating eosinophils with and without a test component and a fungal cell (e.g., an Alternaria cell, a Candida cell, an Aspergillus cell, or a Cladosporium cell), and determining whether the eosinophil localizes to the fungal cell and degranulates in the presence and absence of the test component.

Eosinophils can be purified from the blood of a CRS patient or a normal individual by methods known in the art. Suitable test components include, for example, T cell factors, such as those that can be purified from the supernatant of a T cell cultured with a fungal preparation (e.g., an Alternaria cell extract) in the presence of an APC. Fractions of such T cell supernatants also may be useful test components. Methods of fractionating supernatants and purifying factors are known in the art and include, for example column chromatography (e.g., using a gel exclusion or an ion exchange column) and gel electrophoresis.

Eosinophil localization can be observed by, for example, light microscopy. Alternatively, the interaction between an eosinophil and a fungal cell can be observed by other methods, including coimmunoprecipitation or fluorescent labeling. A particular test component can be identified as an activator of eosinophil fungal attack if eosinophil localization to a fungal cell and degranulation is increased (by, e.g., 1%, 5%, 10%, 50%, 100%, or more) in the presence of the test component as compared to the level of eosinophil attack observed in the absence of the test component.

Fungus-Induced T Cell Activation

In the course of a normal immune response, the binding of ligand (typically the complex of antigen and a major histocompatability molecule on an APC) to a T cell receptor complex on the surface of a T cell initiates intracellular changes. These changes usually lead to proliferation of the T cell and the sequential activation of a network of signaling molecules such as kinases, phosphatases, adaptor proteins, and other signaling molecules (e.g., cytokines) that couple the stimulatory signal received from the T cell receptor to intracellular signaling pathways. As used herein, “fungus-induced T cell activation” refers to the stimulation of cytokine release from a T cell by a factor found in a fungal preparation.

As described below (see Example 1), T cell activation can be achieved by incubating a T cell from a CRS patient with a fungal preparation (e.g., a fungal cell extract or a fungal culture supernatant) in the presence of an APC. T cells can be isolated from a CRS patient or from a normal patient, although T cells from a CRS patient typically are more strongly activated. Alternatively, T cells can be within a PBMC preparation from a subject such as a CRS patient. T cells also can be obtained as an immortalized cell line. The fungal preparation can be from any suitable organism, including but not limited to those listed above. Preparations from Alternaria, Candida, Aspergillus, and Cladisporium are particularly useful. APC can be purified cells (e.g., dendritic cells, macrophages, or B cells) either isolated from a subject or in an immortalized cell line, or APC can be those present within a PBMC preparation that can simultaneously serve as a source of T cells to be activated. Useful lengths of time for incubation and concentrations of fungal preparations can be readily determined by those of ordinary skill in the art.

T cell activation can be measured by, for example, determining the level of cytokine production, cytokine gene expression, or cytokine bioactivity. Measuring levels of interleukin-5 (IL-5), interleukin-13 (IL-13), and/or interferon-gamma (IFN-γ) is particularly useful. The production of these cytokines can be measured at the protein level (e.g., by immunoassay such as western blotting or ELISA), at the mRNA level (e.g., by northern blotting or RT-PCR), or at the activity level (e.g., by bioassays that measure cellular proliferation in response to a particular cytokine produced by a T cell). A number of these methods are established in the art and can be easily performed by one of ordinary skill.

Identifying an Inhibitor of Fungus-Induced T Cell Response

The invention provides methods and materials that can be used to identify an inhibitor of a T cell response to fungus. For example, an inhibitor of fungus-induced T cell activation can be identified by contacting a T cell from a CRS patient with a fungal preparation (e.g., a cell extract or culture supernatant from Alternaria, Candida, Aspergillus, or Cladisporium) and an APC in the presence and absence of a test compound, and measuring cytokine output by the T cell. A test compound can be identified as an inhibitor of a fungus-induced T cell response if cytokine production by the T cell is reduced (e.g., by 1%, 5%, 10%, 50%, or 100%) in the presence of the test compound as compared to cytokine production in the absence of the test compound.

T cells can be obtained from, for example, the blood of a CRS patient, or can be contained within a PBMC isolate from a CRS patient. APC can be similarly obtained (i.e., can be either purified or contained within a PBMC preparation from a CRS patient). Fungal preparations can be commercially obtained or can be prepared by standard laboratory methods. The assessment of cytokine production by measuring IL-5, IL-13, and/or IFN-γ is particularly useful to evaluate a T cell response, and can be conducted at the mRNA level, the protein level or the bioactivity level, as described above. Suitable concentrations of test compounds and times for incubation with T cells can be readily determined by those of ordinary skill in the art.

Identifying a Fungal T Cell Activating Molecule

One or more antigens from fungi (e.g., Alternaria, Candida, and Aspergillus) can stimulate a T cell response, typically in T cells from a CRS patient, such that the T cells release cytokines (e.g., IL-5, IL-13, and IFN-γ). The invention provides methods and materials that can be used to identify the fungal antigens responsible for this T cell response.

The fungal antigens can be polypeptides. The stimulation of cytokine release from T cells in response to one or more fungal antigens typically requires the presence of APC.

A fungal antigen that induces a T cell response can be identified by, for example, incubating (e.g., for 24 to 96 hours) a T cell from a CRS patient with and without a test antigen, and determining the level of cytokine output by the T cell. A particular test antigen can be identified as inducing a T cell response if the level of cytokine production by T cells is increased in the presence of the test antigen as compared to the level observed in the absence of the test antigen. Specificity can be assessed by, for example, evaluating the response of T cells from a normal individual as compared to a CRS patient.

T cells can be obtained from, for example, the blood of a normal individual or a CRS patient, either in purified form or within a PBMC isolate. APC can be similarly obtained. Suitable test antigens include, for example, purified or recombinantly expressed fungal polypeptides or fragments thereof. Test antigens also can be contained within fungal cell extracts or fungal culture supernatants, or fractions thereof. Non-limiting examples of methods for fractionating cellular extracts or culture supernatants include column chromatography (e.g., using a gel exclusion or an ion exchange column) and gel electrophoresis. Fungal cell and culture extracts from, for example, Alternaria, Candida, Aspergillus, and Cladisporium can be particularly useful, as are individual molecules that have been purified from such fungal preparations. Cytokine output determined by, for example, measuring levels of IL-5, IL-13, and/or IFN-γ as described above also is particularly useful.

Animal Models

The invention provides animals having a fungal antigen-induced eosinophilic response, which can be used to identify inhibitors of fungus-induced eosinophilia. Such animals can be pigs, goats, sheep, cows, horses, rabbits, rodents such as rats, guinea pigs and mice, and non-human primates such as baboons, monkeys and chimpanzees. For example, wild mice strains or laboratory mice strains (e.g., Blab, black6, or nude mice) can be used to make an animal model having fungus-induced eosinophilia. Suitable mice can be congenic, transgenic, or “knockout” mice. The animal models can be animals that are housed within cages in a research animal facility.

The invention also provides populations of animal models (e.g., 3, 4, 5, 6, 7, 8, 9, 10, 20, 50, 100, or more animals) having fungus-induced eosinophilia. Such populations can contain animals from the same species or strain. The invention also provides a combination of two or more animal populations. One animal population can be animals having fungus-induced eosinophilia, and another population can be similar animals not having fungus-induced eosinophilia Alternatively, one animal population can be animals having fungus-induced eosinophilia, and another population can be similar animals having eosinophilia but not fungus-induced eosinophilia. Such populations and combinations of populations can be used to test compounds for the ability to inhibit fungus-induced eosinophilia.

The animal models provided herein can be used to evaluate immune responses in a variety of conditions such as asthma, sinusitis, otitis, and Crohn's disease. Animals in which an immune response has been induced by challenge with one or more fungal antigens can be used to screen for inhibitors of such an immune response. Compounds that can be screened include, by way of example, a biological macromolecule such as an oligonucleotide or a polypeptide (e.g., enzymes, proteins, antibodies, or carbohydrates), a chemical compound, a mixture of chemical compounds, or an extract or fraction isolated from bacterial, plant, fungal or animal matter. In one embodiment, potential treatment compounds are identified by administering a test compound to an animal (e.g., mouse) having fungus-induced eosinophilia and determining whether or not the test compound reduced the level of fungus-induced eosinophilia in that animal as compared to the level in a control animal that did not receive the test compound.

The invention also provides methods for making an animal having fungus-induced eosinophilia. Such methods can involve administering one or more fungal antigens (e.g., an Alternaria extract) to an animal. The animal can be a previously sensitized animal or a naïve animal (e.g., an animal not previous given the fungal antigen or antigens). For example, the animals can be sensitized with one or more fungal antigens prior to the challenge that results in fungus-induced eosinophilia. Animals can be sensitized and challenged with a fungal antigens using any one of a number of methods. For example, fungal antigens can be administered to an animal intraperitoneally, nasally (e.g., by aerosolization or installation), systemically, or orally. In other words, fungal antigens can be administered to any part of the animal (e.g., nose, lungs, ear, muscle, skin, foot, or stomach). In some embodiments, one or more fungal antigens are administered to a portion of the airways of an animal. Any amount (e.g., from about 5 μg to about 100 g) of fungal antigen can be used. For example, 25 μg of a fungal extract can be administered to an animal to induce fungus-induced eosinophilia. The fungus-induced eosinophilia can be present in any tissue of the animal (e.g., lung, sinus, colon, or ear tissue).

Any fungal antigen can be used to make such animals. For example, fungal spores, fungal extracts, or conditioned medium (e.g., medium in which fungal cells were grown) can be used as an antigen to induce an eosinophilic response. Antigens can be obtained from a number of different fungi, including but not limited to, Absidia, Aspergillus flavus, Aspergillus fumigatus, Aspergillus glaucus, Aspergillus nidulans, Aspergillus versicolor, Alternaria, Basidiobolus, Bipolaris, Candida albicans, Candida lypolytica, Candida parapsilosis, Cladosporium, Conidiobolus, Cunninahamella, Curvularia, Dreschlera, Exserohilum, Fusarium, Malbranchia, Paecilomvces, Penicillium, Pseudallescheria, Rhizopus, Schizophylum, Sporothrix, Acremonium, Arachniotus citrinus, Aurobasidioum, Beauveria, Chaetomium, Chryosporium, Epicoccum, Exophilia jeanselmei, Geotrichum, Oidiodendron, Phoma, Pithomyces, Rhinocladiella, Rhodoturula, Sagrahamala, Scolebasidium, Scopulariopsis, Ustilago, Trichoderma, and Zygomycete. A list of additional fungal organisms that can be used to generate antigen can be found in most taxonomic mycology textbooks.

Administration of a fungal antigen to an animal can induce a measurable eosinophilic response in the animal. The eosinophilic response can be measured using a number of different methods. For example, standard histochemical and immunohistochemical techniques can be used to assess the presence or absence of eosinophils in tissues (e.g., lung tissue) from a treated animal.

The invention will be further described in the following examples, which do not limit the scope of the invention described in the claims.

### EXAMPLES

### Example 1

### Lymphocytes from CRS Patients Produced IL-5 in Response to Fungal Antigens

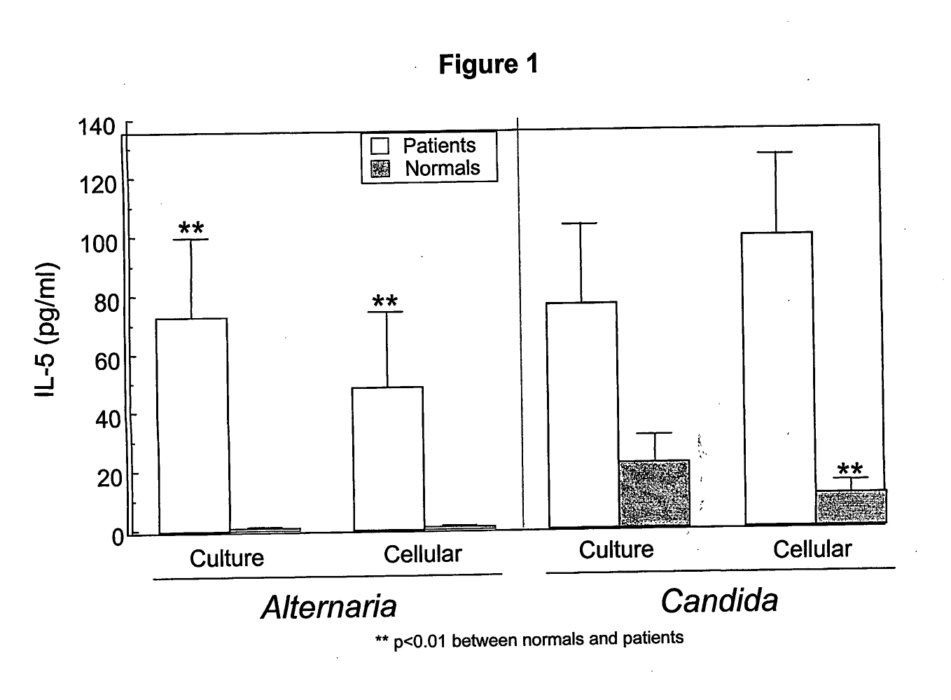

The lymphocyte response to fungal products was examined. PBMC were isolated from CRS patients or from normal individuals, suspended in RPMI 1640 medium supplemented with 10% bovine calf serum, and incubated for 3 days with 50 μg/mL of extracts from five fungal species most frequently isolated from nasal cavities of patients (Alternaria alternata, Candida albicans, Aspergillus versicolor, Cladosporium herbarum, and Penicillium notatum). All extracts were purchased from Greer Laboratories (Lenoir, N.C.). Extracts from both fungal culture medium (“culture”) and fungal organisms (“cellular”) were tested because of potential differences in antigenicity. After incubation of the fungal extracts with the PBMC from patients or normal individuals, cell-free supernatants were collected, and the amount of IL-5 in each sample was measured by ELISA (Endogen, Inc., Woburn, Mass.). IL-5 was chosen as a marker of the lymphocytic response because this cytokine is implicated in the pathophysiology of eosinophilic inflammation and is commonly detected in the sinus mucosa of CRS patients. PBMC from CRS patients produced significant amounts of IL-5 when incubated with cellular or culture extracts from Alternaria and Candida (FIG. 1). Aspergillus cellular extract and Cladosporium culture extract also induced some IL-5 production. In contrast, extracts from Penicillium did not stimulate IL-5 production, suggesting specificity in the cellular response. These results indicate that fungal antigens can stimulate IL-5 production in PBMC from CRS patients.

Although fungi appear to colonize the nasal cavities of both CRS patients and normal subjects, only CRS patients have disease. The results depicted in FIG. 1 indicate that patients' lymphocytes respond to fungal antigens differently than lymphocytes from healthy individuals. As shown in FIG. 1, PBMC from 18 CRS patients produced detectable amounts of IL-5 when incubated with Alternaria extracts. In contrast, none of the PBMC from the 11 normal subjects tested produced an IL-5 response to either the culture or cellular Alternaria extract (p<0.01 between normal subjects and CRS patients). Furthermore, when incubated with Candida extracts, PBMC from CRS patients again produced significantly more IL-5 than PBMC from normal subjects p<0.01 for the cellular extract). Similar observations were found with Cladosporium cellular and culture extracts, although the overall response to Cladosporium antigens was weaker. When PBMC were incubated with Aspergillus cellular and culture extracts, IL-5 also was detectable in cells from about 70% of the CRS patients but was undetectable in cells from normal subjects. No IL-5 production was observed with patient or normal PBMC cultured with Penicillium cellular or culture extracts, and no IL-5 was detected in patient or normal PBMC cultured in the absence of fungal extracts. These results indicate that PBMC from CRS patients are unique in their IL-5 responses to fungal antigens. Furthermore, among the various fungal species tested, Alternaria antigens seemed to most distinctly differentiate between the lymphocytic response of a patient and that of a normal individual.

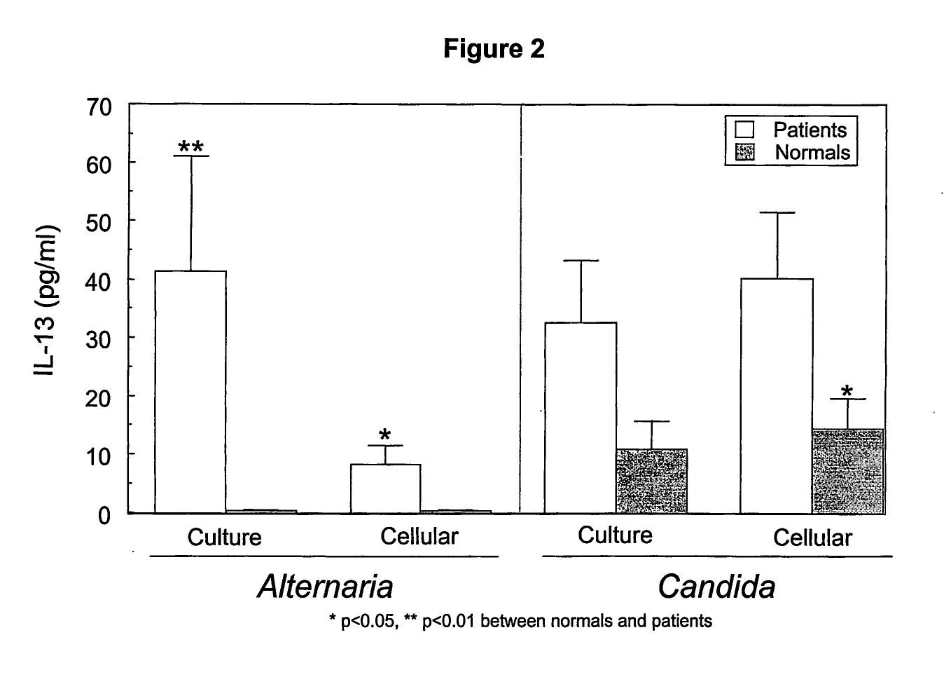

Similar results were obtained when IL-13 production was measured, as PBMC from CRS patients produced significantly more IL-13 than PBMC from normal subjects in the presence of fungal extracts (FIG. 2). IL-13 levels were measured by ELISA (Endogen, Inc., Woburn, Mass.). The difference between CRS patients and normal subjects was greatest when PBMC were treated with Alternaria culture supernatants (D<0.01), although statistical significance also was achieved with Alternaria and Candida cellular extracts (p<0.05). In other experiments, the output of interferon-γ (IFN-γ) as measured by ELISA (Endogen, Inc., Woburn, Mass.) also was significantly increased by treatment of CRS patient PBMC with fungal preparations, although the results were more variable.

### Example 2

### Alternaria Antigens Induce Activation and Degranulation of Eosinophils

The ability to induce eosinophilic degranulation was measured by EDN release. Human eosinophils were isolated from normal volunteers and volunteers reporting past allergies, hay fever, and/or asthma by Percoll density gradient centrifugation and magnetic cell sorting using MACS anti-CD16 microbeads as described by Hansel et al. (1991, J. Immunol. Methods, 145:105). Briefly, after peripheral blood was overlaid on an isotonic Percoll solution (1.084 g/ml; Sigma), the blood was centrifuged at 1000×g for 30 minutes at 4° C. Mononuclear cells at the interface were removed, and erythrocytes in the sediment were lysed by two cycles of hypotonic water lysis. Isolated granulocytes were washed twice with PIPES buffer (25 mM PIPES, 50 mM NaCl, 5 mM KCl, 25 mM NaOH, 5.4 mM glucose, pH 7.4) containing 1% bovine calf serum (Hyclone Laboratories; Logan, Utah). Cells were then incubated with an equal volume of anti-CD16-conjugated magnetic beads (Miltenyi Biotec, Auburn, Calif.) for 30 minutes at 4° C. with occasional gentle mixing. After 30 minutes of incubation on ice, cells were loaded onto a separation column positioned in the strong magnetic field of the MACS. Cells were eluted three times with 5 mL of PIPES buffer containing 1% bovine calf serum. The purity of eosinophils, as counted by Randolph's stain, was regularly >98%. Purified eosinophils were used immediately for experiments.

To monitor eosinophil degranulation, 200 μL of freshly isolated eosinophils were resuspended in HBSS with 25 mM HEPES and 0.01% gelatin at 5×10⁵ cells/mL and incubated with the a particular fungal extract. After incubation for 3 hours at 37° C. in a 5% CO₂ atmosphere, cell-free supernatants from each well were collected and stored at −20° C. To quantitate eosinophil degranulation, the concentration of one of the eosinophil granule proteins in the sample supernatants, EDN, was measured by specific RIA, as described previously (Horie & Kita, 1994, J. Immunol., 152:5457). The RIA is a double-Ab competition assay in which radioiodinated EDN, rabbit anti-EDN Ab, and burro anti-rabbit IgG are used (Abu-Ghazaleh et al., 1989, J. Immunol., 142:2393). Sensitivity of the kit was 2 ng/mL. Total cellular EDN was measured in parallel supernatants from cells lysed with Nonidet P-40 detergent (Sigma Chemical Co.). All experiments were conducted in duplicate.

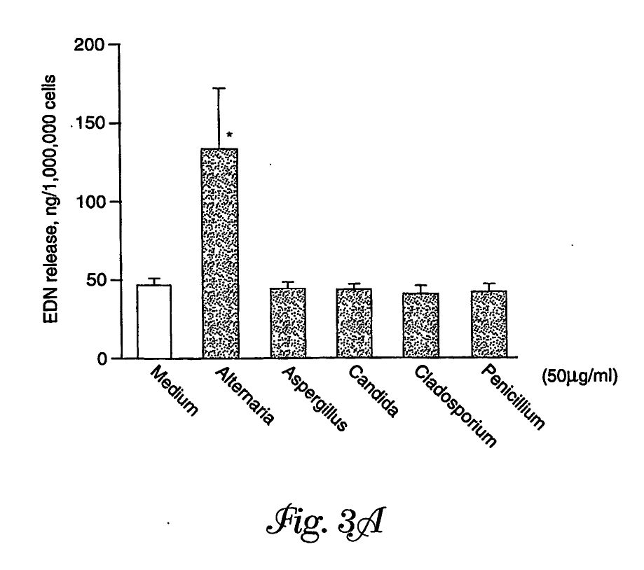

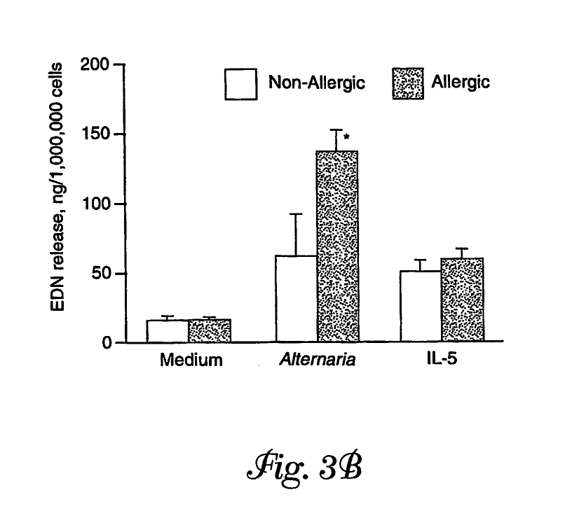

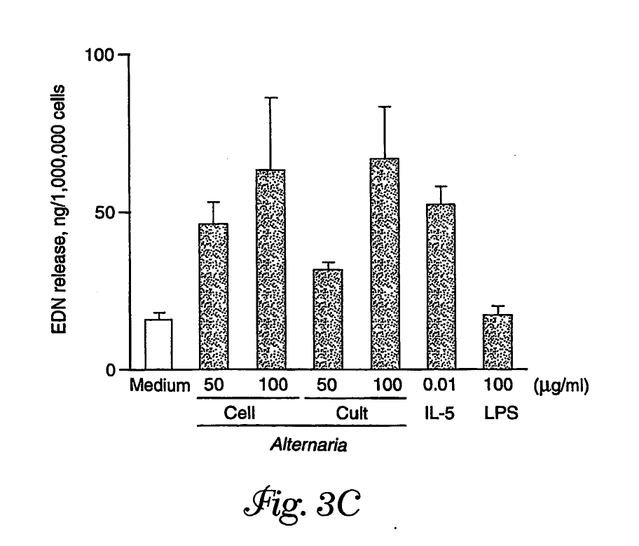

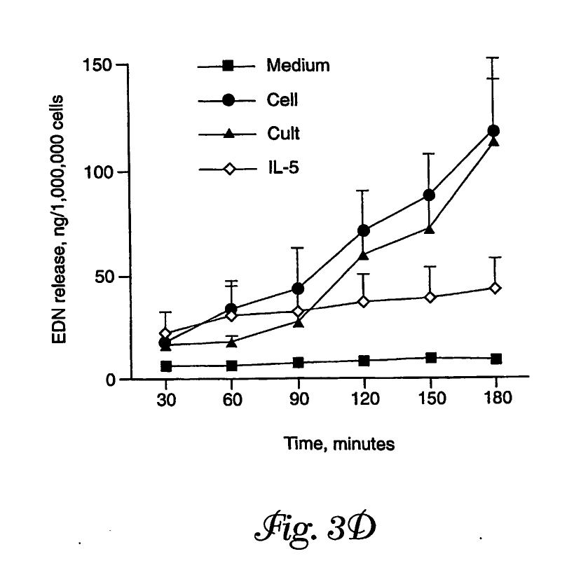

Purified eosinophils from patients reporting allergies were incubated under the conditions described above with 50 μg/mL of culture extract from Alternaria, Aspergillus, Candida, Cladosporium, or Penicillium fungi, and EDN was measured as described above. Alternaria extract was the only fungi to produce significant eosinophilic degranulation (p<0.05), as measured by EDN release (FIG. 3A).

To investigate whether the magnitude of the eosinophil response to Alternaria is different for patients exhibiting allergies than for normal individuals, the release of EDN was compared between allergy patients and normal individuals. Eosinophils were incubated in the presence of 100 μg/mL of Alternaria culture extracts or 10 ng/mL of IL-5. Interestingly, significantly more Alternaria-induced EDN release was observed for eosinophils obtained from allergy patients than for eosinophils obtained from normal individuals (p<0.05). There was no difference in the amount of degranulation observed in eosinophils obtained from allergy patients and normal individuals when those cells were stimulated with IL-5 (FIG. 3B).

Alternaria extracts also were tested at different concentrations (FIG. 3C). At 50 μg/mL, Alternaria cellular and culture extracts induced release of 45.2±7.0 and 30.5±2.1 ng EDN/10⁶ cells, respectively. At 100 μg/mL, Alternaria cellular and culture extracts induced release of 63.5±23.2 and 67.1±16.7 ng EDN/10⁶ cells, respectively. These values correspond to 127.7±44.3 and 140.2±37.5 percent of the EDN release induced by 10 ng/ml IL-5, respectively. They also correspond to 8.6±2.9 and 9.2±2.0 percent of the total EDN contents, respectively (mean±SEM, n=9). At 10 ng/mL, IL-5 induced release of 52.1±6.5 ng EDN/10⁶ cells. In contrast, bacterial LPS (100 μg/mL) induced little, if any, eosinophil degranulation, suggesting that the Alternaria-induced degranulation of eosinophils was not due to LPS contamination.

Additionally, there was a time dependent increase in eosinophil degranulation stimulated by Alternaria extracts (FIG. 3D). Incubation of eosinophils with 100 μg/mL of Alternaria cellular or culture extracts resulted in a faster rate of degranulation than was observed with 10 ng/mL of IL-5. Eosinophils degranulated about 2.5 times faster in the presence of Alternaria extracts than in the presence of IL-5.

The effects of Alternaria proteins (extracted by ammonium sulfate precipitation) and viable Alternaria (purchased from American Type Culture Collection (ATCC; Manassas, Va.)) also were evaluated on human eosinophils. These experiments confirmed that eosinophils degranulated in the presence of Alternaria (14.5% and 11.1% of total EDN release, respectively).

To examine whether Alternaria induces eosinophil degranulation of other eosinophil components, MBP release was measured by 2-site immunoradiometric assay, as described previously (Wagner et al., 1993, Placenta, 14:671). Because MBP exhibits attachment to plastic tubes and can be difficult to detect in the supernatant at neutral pH, MBP amounts in the pellets of cells lysed by Nonidet P-40 detergent after exposure to Alternaria extracts was measured, then the percent of total MBP was calculated according to the following equation: percent of total MBP=(total MBP in lysate of eosinophils before incubation−MBP in lysate after stimulation)/total MBP in lysate of eosinophils before incubation×100. Results indicated that 47.2% and 57.7% of total MBP was released from eosinophils when the eosinophils were incubated with culture extracts of Alternaria (100 μg/mL) (n=2).

### Example 3

### Characteristics of Alternaria Antigens

The mechanism of the eosinophil degranulation response to Alternaria antigens was examined. To test whether Alternaria proteases are involved in eosinophil activation, Alternaria extracts either were pretreated with a cocktail of protease inhibitors (Complete™, Roche Molecular Biochemicals, Indianapolis, Ind.) or were not pretreated (control). Eosinophils then were incubated with the pretreated or control Alternaria extracts for 4 hours, and EDN release was measured. The cocktail of protease inhibitors produced an 80% inhibition of Alternaria-induced eosinophil degranulation. In contrast, the same protease treatment did not affect phorbol myristate acetate (PMA)-induced degranulation. Eosinophil degranulation induced by 1 μM platelet activating factor also was not affected by protease inhibitors (−5%±4% inhibition, mean±range, n=2). These findings suggest that protease activity in Alternaria extracts is necessary for the induction of eosinophil degranulation. Furthermore, a specific and irreversible serine protease inhibitor, AEBSF, inhibited the ability of Alternaria extracts to induce degranulation by 89%, suggesting that one or more serine proteases may play a role in eosinophil degranulation.

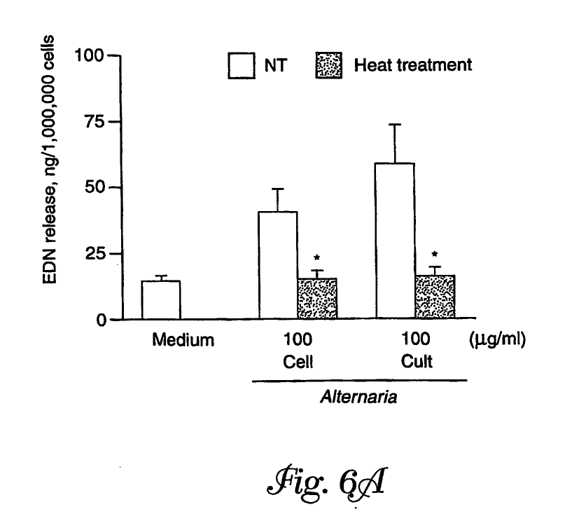

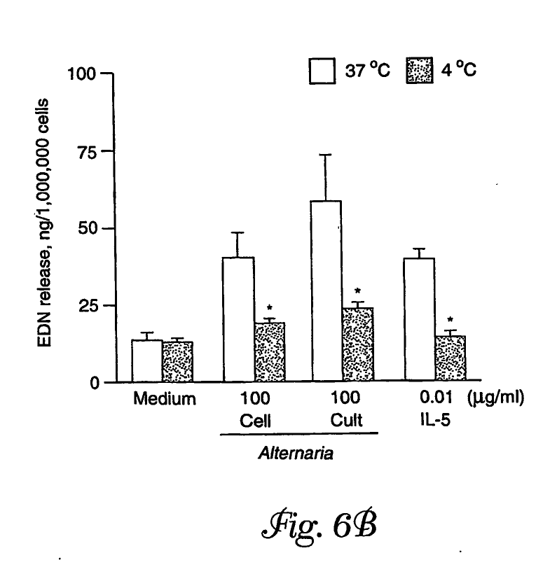

In addition, Alternaria culture extracts were filtered at 4° C. through a hydrophilic membrane having a 50-kDa cut-off size (Centricon Centrifugal Filter Devices, Millipore Corporation, Bedford, Mass.). Based on eosinophil degranulation and using EDN release as a marker, the stimulatory activity in the Alternaria culture extract did not exist in the filtrate, but was present in the supernatant. This result indicates that stimulatory activity of Alternaria extracts exists in a fraction of >50 kDa. The activity was completely abolished by heat-treatment of the Alternaria extract at 100° C. for 10 minutes (p<0.05; FIG. 6A), suggesting that the stimulatory activity of Alternaria is likely a result of one or more protein or glycoprotein antigens.

### Example 4

### Microscopy of Eosinophils

Light microscopy indicated that eosinophils that were activated and stimulated with cytokines from PBMC of CRS patients were able to cluster around fungi and degranulate, thus destroying the fungal bodies (FIG. 4). Normal eosinophils that were not activated, however, did not degranulate on fungi (FIG. 5). Large amounts of eosinophil granule proteins (e.g., MBP) normally are released from eosinophils during degranulation, and MBP typically is present in damaging concentrations in the mucin of asthmatics and CRS patients. Antibody staining of purified eosinophils and fungi revealed that most of the cells had released MBP and had a void image. The release of free MBP has not previously been demonstrated successfully in vitro.

The morphology of the eosinophils was examined by electron microscopy. Isolated eosinophils were incubated in HBSS buffer for 2 hours at 37° C. under 5% CO₂ in microcentrifuge tubes pre-coated with 1% HSA. The cell pellets were fixed in 4% formaldehyde and 1% glutaraldehyde in 0.1 mol/L phosphate buffer (pH 7.2) for 16 hours and centrifuged at 400×g for 5 minutes. The cell pellet was rinsed 3 times in 0.1 mol/L phosphate buffer (pH 7.2) and post-fixed with 1% phosphate-buffered osmium tetroxide for 60 minutes, rinsed in 3 changes of water, and stained en bloc with 2% aqueous uranyl acetate at 60° C. The cells were rinsed briefly in distilled water and dehydrated in graded concentrations of ethanol. After combining Spurr resin and ethanol 1:1 and 3:1, the cells were infiltrated with each mixture for 60 minutes and resuspended in fresh Spurr resin overnight. The next day, the cells were embedded in polyethylene capsules. Thin sections with a gold interference color were collected on uncoated copper grids (200 mesh) and examined with a transmission electron microscope (JOEL 1200, JOEL USA, Peabody, Mass.).

When incubated with medium alone at 37° C. for 3 hours, most eosinophils maintained cytoplasmic granules with characteristic core and matrix structures and intact plasma membranes. Following 3 hours of incubation with 100 μg/mL culture extract of Alternaria, electron microscopy revealed granule fusion and electron-lucent granule cores and matrices, while the plasma membrane remained grossly intact. To examine whether the eosinophil degranulation induced by Alternaria is mediated by the cell's physiologic activation mechanisms or the result of cytolytic effects of the Alternaria, the temperature dependency of eosinophil degranulation was examined. Incubation of eosinophils with Alternaria cellular extract, Alternaria culture extract, or IL-5 at 4° C. for 3 hours abrogated EDN release induced by both Alternaria cellular and culture extracts, as well as EDN release induced by IL-5 (p<0.05; FIG. 6B). Thus, Alternaria likely induced regulated, or exocytotic eosinophil degranulation, but did not induce cytolysis.

### Example 5

### Effect of Cytochalasin B and Anti-CD18 mAb on Alternaria-Induced Eosinophil Degranulation

To characterize the eosinophil degranulation induced by Alternaria, cytochalasin B was used. Cytochalasin B is known to inhibit the assembly of microtubes. It was previously reported that cytochalasin B inhibits eosinophil degranulation stimulated with various physiologic agonists such as cytokines (e.g., IL-5) and lipid mediators (e.g., platelet activating factor).

To examine the effect of cytochalasin B in the eosinophil response, eosinophils were preincubated with or without cytochalasin B (5 μg/mL) for 15 minutes at 37° C. After preincubation, eosinophils were exposed to either Alternaria culture extract (100 μg/mL) or IL-5 (10 ng/mL) for 3 hours at 37° C., and EDN release from eosinophils was measured.

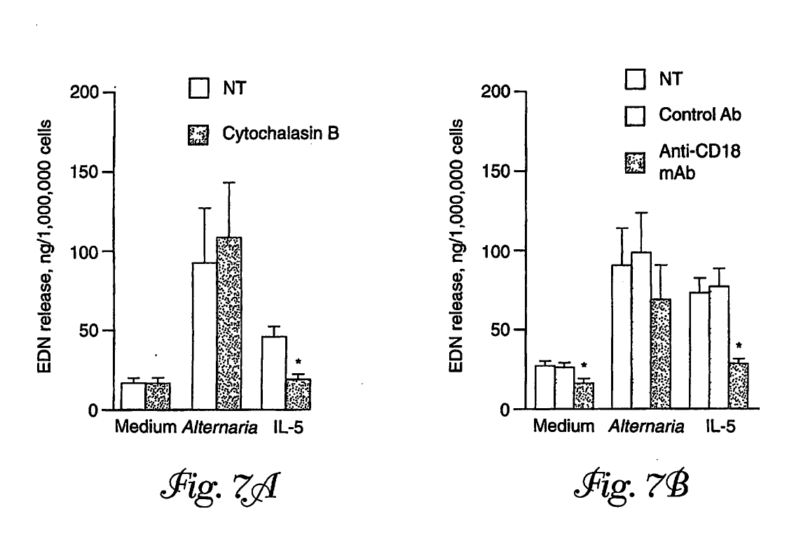

In contrast to the IL-5-induced degranulation response, the eosinophil degranulation induced by Alternaria culture extract was not affected by pretreatment of cells with cytochalasin B (FIG. 7A). On the other hand, the IL-5-induced degranulation response was inhibited by pretreatment of eosinophils with cytochalasin B (p<0.05; FIG. 7A).

Next, to examine the role of adhesion processes in eosinophil degranulation induced by stimulants such as IL-5 or platelet activating factor, the effect of pretreating cells with a mAb against an integrin-family receptor, αMβ2 (Mac-1, CD11b/CD18), was examined. Anti-CD18 mAb, which exhibits binding affinity for the β subunit of the Mac-1 heterodimer and thereby blocks cell adhesion, was used. A rat IgG1 antibody (10 μg/mL) was used as a control.

Results indicated that at 10 μg/mL, anti-CD18 mAb failed to inhibit Alternaria-induced eosinophil degranulation (FIG. 7B). In addition, results also indicated that the anti-CD18 mAb inhibited IL-5-induced degranulation p<0.05; FIG. 7B). It was further noted that anti-CD18 mAB inhibited the amount of eosinophilic degranulation in the control treatment (medium, FIG. 7B). Although not bound by any particular mechanism, it is proposed that eosinophils adhere to the plastic tube, which stimulates degranulation. Therefore, anti-CD18 mAb inhibited this adhesion-induced degranulation in the control.

In similar experiments, various concentrations of EDTA, cyclosporine A (a calcineurin inhibitor), FK-506 (a calcineurin inhibitor), Calpeptin (a calcineurin inhibitor), SB203580 (a p38 inhibitor), PD98059 (an inhibitor of extracellular signal regulated kinase 2 (Erk2)), Genistein (an inhibitor of protein tyrosine kinase (PTK)), Herbmycin (a PTK inhibitor), Wortmannin (an inhibitor of phosphatidyl inositol 3-kinase (PI3 kinase)), Go 6976 (an inhibitor of the α and β subunits of protein kinase C (PKC)), Rottelerin (an inhibitor of the 6 subunit of PKC), GF109203X (an inhibitor of the α, β, δ, and ε subunits of PKC), and CV6209 (a platelet-activating factor (PAF) receptor antagonist) were used to examine their effect on Alternaria- and IL-5-induced eosinophil degranulation. In general, EDTA completely inhibited Alternaria-induced eosinophil degranulation. CV6209 also inhibited Alternaria-induced degranulation in a concentration-dependent manner with an IC₅₀ value of about 1 μM. The other compounds listed above had no effect on Alternaria-induced degranulation. In general, EDTA, SB203580, Genistein, Herbmycin, Wortmannin, Go 6976, and Rottelerin completely inhibited the IL-5-induced eosinophil degranulation response; calpeptin, PD98059, and GF109203X partially inhibited the eosinophilic degranulation response to IL-5; and cyclosporine A and FK-506 had no effect on the IL-5-induced degranulation response.

### Example 6

### Calcium Requirement for Alternaria-Induced Eosinophil Degranulation

Stimulation of eosinophils with chemoattractant molecules leads to elevation of intracellular levels of calcium, which is implicated in eosinophil degranulation. To investigate the intracellular mechanism of eosinophil degranulation induced by Alternaria, the requirement for calcium was examined.

Real time changes in cytosolic free Ca²⁺ (intracellular Ca²⁺ concentration ([Ca²⁺]i)) were measured in a flow cytometer using the fluorescent calcium indicator indo-1 (Rabinovitch et al., 1986, J. Immunol., 137:952; Grynkiewicz et al., 1985, J. Biol Chem., 260:3440). Indicator was introduced into cells by incubating 1 mL of eosinophils at a concentration of 1 to 2×10⁶ cells/mL with 3 μM indo-1/AM (Molecular Probes, Eugene, Oreg.) in phenol red-free RPMI 1640 medium supplemented with 10% alpha calf serum and 10 mM HEPES for 30 minutes at 37° C. After washing, cells were suspended in RPMI with 0.1% HSA and 10 mM HEPES at 1×10⁶ cells/mL. To measure [Ca²⁺]i, cells were stimulated with 100 μg/mL Alternaria culture extract or 10 ng/mL IL-5, and fluorescence was analyzed by a FACS analyzer equipped with an ion-argon laser (Becton Dickinson). [Ca²⁺]i was monitored on the basis of the ratio of the fluorescence of the calcium-bound indo-1 emission (401 nm) and the free indo-1 emission (475 nm). In addition, the [Ca²⁺] in the medium can be manipulated to determine the effect on [Ca²⁺]i.

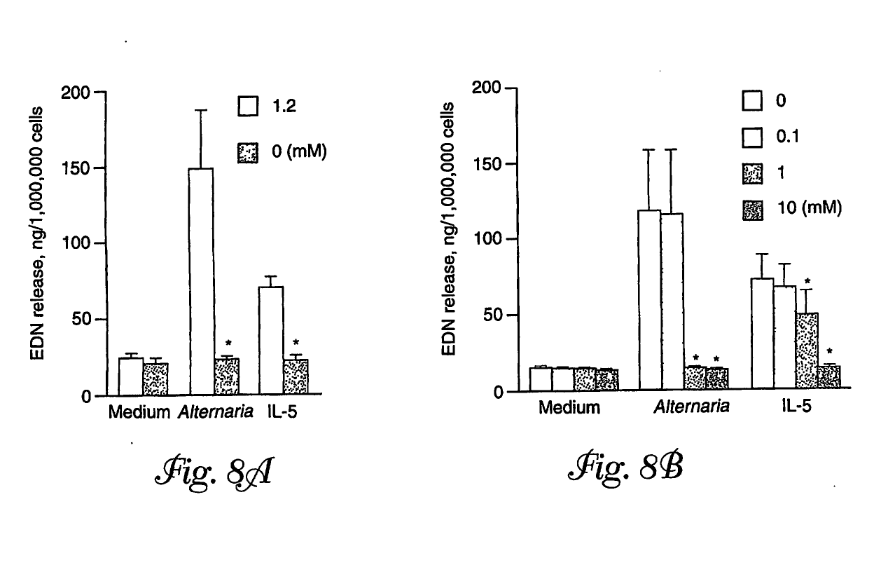

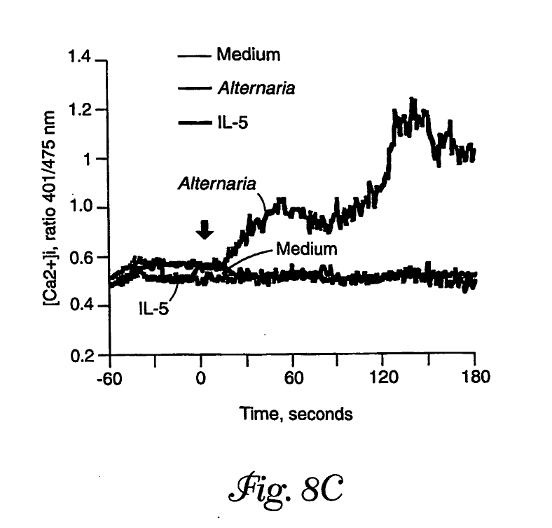

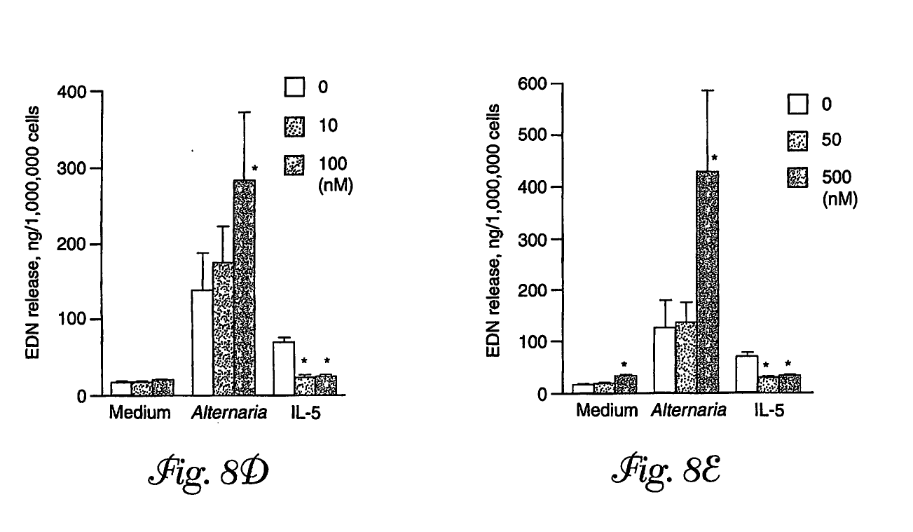

As shown in FIG. 8A, eosinophil degranulation induced by Alternaria or IL-5 was sensitive to the concentration of calcium in the medium. A calcium concentration in the medium of 1.2 mM did not inhibit the Alternaria-induced eosinophil degranulation, however, degranulation was inhibited completely by lowering the calcium concentration in the medium to 0 mM (p<0.05).

Next, the effect of a Ca²⁺ chelator, EGTA, on Alternaria- or IL-5-induced eosinophil degranulation was investigated. As shown in FIG. 8B, pretreatment of cells with EGTA for 15 minutes blocked eosinophil degranulation induced by Alternaria and IL-5 in a concentration-dependent manner. Interestingly, 1 mM EGTA almost completely inhibited the eosinophil degranulation induced by Alternaria (p<0.05; FIG. 8B). In contrast, the IL-5-induced degranulation response was only partially inhibited by 1 mM EGTA (p<0.05; FIG. 8B). 10 mM EGTA almost completely inhibited the Alternaria-induced and IL-5-induced eosinophil degranulation (FIG. 8B). These findings suggest that eosinophil degranulation induced by Alternaria, and to a lesser extent by IL-5, is highly dependent on extracellular calcium.

To further investigate the effect of calcium on eosinophil degranulation, changes in [Ca²⁺]i over time were measured in eosinophils loaded with calcium-sensitive fluorescent dye. Stimulating eosinophils with 100 μg/mL Alternaria culture extract at 37° C. produced an increase in [Ca²⁺]i that became evident almost immediately after addition of the extract (FIG. 8C). In contrast, no elevation of [Ca²⁺]i was observed in eosinophils stimulated with 10 ng/mL IL-5 (FIG. 8C). These findings suggest that calcium may play a role in the degranulation mechanism of Alternaria-induced eosinophils.

Because increases in [Ca²⁺]i would be expected during directed migration of eosinophils into inflammatory sites, the priming effect of well-defined agonists that induce [Ca²⁺]i mobilization on Alternaria-induced eosinophil degranulation was investigated using ionomycin and thapsigargin. Ionomycin is a calcium ionophore. Thapsigargin elevates [Ca²⁺]i from intracellular stores by inhibiting the endoplasmic reticulum Ca²⁺-ATPase, thus allowing influx to the cytoplasm.

Eosinophils were pre-treated with various concentrations of ionomycin (FIG. 8D) or thapsigargin (FIG. 8E) for 15 minutes, and then exposed to Alternaria culture extract (100 μg/mL) or IL-5 (10 ng/mL) for 3 hours at 37° C. Alternaria-induced eosinophil degranulation was synergistically increased when in the presence of ionomycin (FIG. 8D) or thapsigargin (FIG. 8E). In contrast, IL-5-induced eosinophil degranulation was significantly inhibited in the presence of either ionomycin (FIG. 8D) or thapsigargin (FIG. 8E). These findings suggest that Alternaria, in combination with ionomycin or thapsigargin, stimulate eosinophil degranulation in a cooperative manner.

### Example 7

### Effects of Alternaria on IL-8 Production by Eosinophils

Human peripheral blood eosinophils can produce and release proinflammtory cytokines including IL-8. Eosinophils were incubated with 50 μg/mL or 100 μg/mL of Alternaria, or with 0.01 μg/mL IL-5, and the amount of IL-8 was measured. Purified eosinophils (1 mL at 1×10⁶ cells/mL) were cultured in RPMI 1640 medium supplemented with 10% BCS in the presence of Alternaria or IL-5 using 48-well tissue culture plates (Costar, Cambridge, Mass.). After incubating the eosinophils with Alternaria or IL-5 for 24 hours at 37° C. in an atmosphere of 5% CO₂, cell free supernatants were collected and frozen at −20° C. The concentrations of IL-8 in the supernatants were measured using an ELISA kit (Quantikine IL-8 Immunoassay Kit, R&D Systems, Minneapolis, Minn.). The threshold sensitivity of the kit was 4 pg/mL.

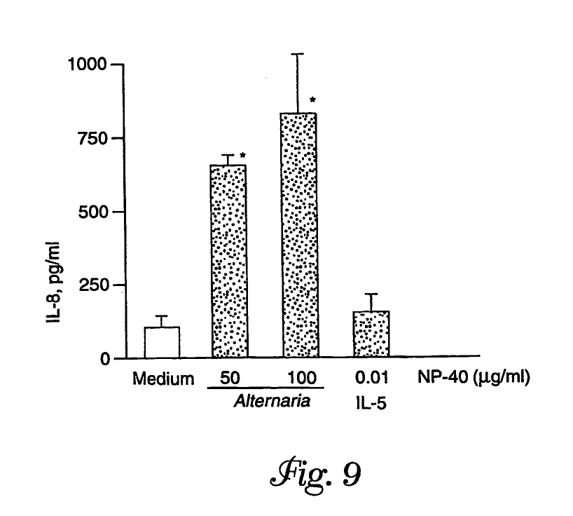

Culture extracts of Alternaria at either concentration induced significant increases in IL-8 production from eosinophils (p<0.05; FIG. 9). Eosinophils treated with IL-5 produced very little IL-8 (FIG. 9). No IL-8 was detected in a 0.5% Nonidet P-40 eosinophil lysate (FIG. 9), indicating that the IL-8 released into the supernatants in the presence of Alternaria was synthesized by eosinophils de novo.

### Example 8

### Effect of Glucocorticoids on the Eosinophilic Response

PBMC were isolated from 30 mL of blood drawn from patients with chronic sinusitis, bronchial asthma, or normal individuals by density centrifugation using Histopaque®. Cells were then suspended in RPMI 1640 medium supplemented with 10% fetal bovine serum at 2×10⁶ cells/mL and incubated for 3 days at 37° C. in 96-well tissue culture plates with medium alone, 25 μg/mL Alternaria cellular extract, 25 μg/mL Dermatophagoides Pteronyssinus (Der P; Greer Laboratories; a mite allergen), or 5 μg/mL Concanavalin A. Since Concanavalin A is a very potent inducer, supernatants of PBMC were diluted 50-fold before they were analyzed for the presence of IL-5.

To examine the effects of glucocorticoids on the eosinophilic response, fluticasone propionate (10⁻¹⁰ M to 10⁻⁶ M, GlaxoSmithKline) was added to the culture. After incubation, cell-free supernatants were collected and analyzed for IL-5 concentrations using an ELISA assay (Endogen, Inc.). In the experiments with Concanavalin A, the amount of IL-5 determined by ELISA was multiplied by 50 to account for the dilution factor.

Results indicated that IL-5 production in Alternaria-induced PBMC was significantly inhibited at the lowest concentration of fluticasone propionate (10⁻¹⁰ M). Similarly, IL-5 production in the presence of Der P and Concanavalin A was inhibited by fluticasone propionate (₁₀—O M). These findings suggest that a glucocorticoid effectively inhibits Alternaria-induced IL-5 production by PBMC as well as IL-5 production induced by DerP or Concanavalin A. Data from a representative asthma patient is shown in Table 1.

TABLE 1
Effect of fluticasone propionate on IL-5 production by eosinophils.

| | | | | Cellular | | | | | | | | | | | |
|---|---|---|---|---|---|---|---|---|---|---|---|---|---|---|---|
| | | | | Alternaria | | | | | | | | | | | |
| | | | | Extract | | | Der P | | | Con A | | | | | |
| Flutica- | Medium | | | (25 μg/mL) | | | (25 μg/mL) | | | (5 μg/mL) | | | | | visual |
| sone | 1 | 2 | mean | 1 | 2 | mean | 1 | 2 | mean | 1 | 2 | 1*50 | 2*50 | mean | ConA |
| medium | 4.2 | 1.2 | 2.7 | 11.0 | 12.4 | 11.7 | 20.1 | 15.2 | 17.6 | 6.8 | 4.0 | 342 | 200 | 271 | ++++ |
| 10⁻¹⁰ M | 1.3 | 0.3 | 0.8 | 1.4 | 3.9 | 2.6 | nd | 0.9 | 0.9 | 1.7 | 2.3 | 87 | 115 | 101 | +++ |
| 10⁻⁹ M | nd | nd | nd | 0.4 | nd | nd | nd | nd | nd | nd | nd | nd | nd | nd | + |
| 10⁻⁸ M | nd | nd | nd | nd | nd | nd | nd | nd | nd | nd | nd | nd | nd | nd | + |
| 10⁻⁷ M | nd | nd | nd | nd | nd | nd | nd | nd | nd | nd | nd | nd | nd | nd | + |
| 10⁻⁶ M | nd | nd | nd | nd | nd | nd | nd | nd | nd | nd | nd | nd | nd | nd | |

nd, not determined.

### Example 9

### The Eosinophilic Response In Vivo in Mice

Mice were sensitized at day 14 with 20 μg/dose OVA or 100 μg/dose Alternaria extracts (cellular or culture) by intraperitoneal injection of the antigens followed by intranasal challenge with the appropriate antigen at 250 μg/dose on days 28, 30, and 32.

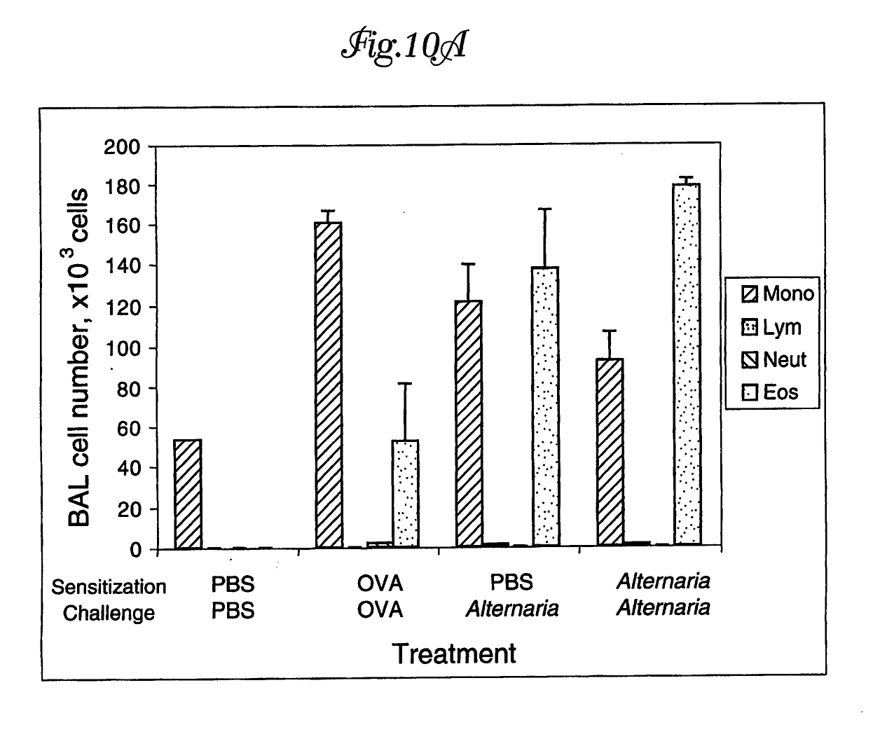

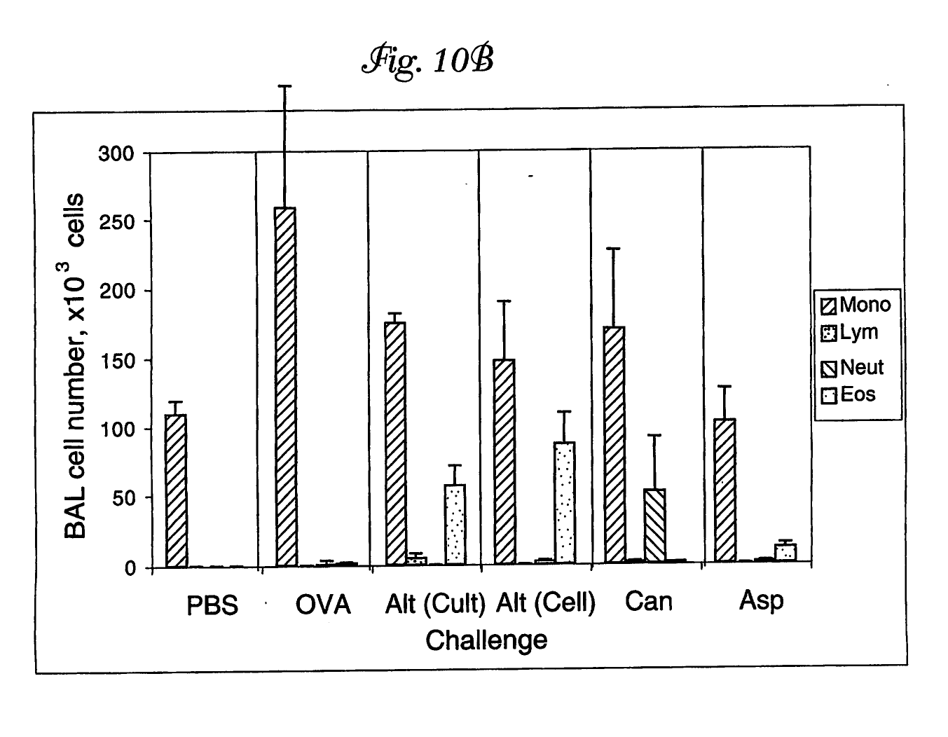

Mice sensitized with OVA or Alternaria culture extract and challenged with OVA or Alternaria culture extract, respectively, developed airway eosinophilia by day 34 (FIG. 10A). Eosinophils were detected in mice by collecting BAL fluids, isolating the cells using a cytospin preparation, and staining the cells with DiffQuick stain (DADE Behring, Inc., Newark, Del.). Interestingly, it was found that non-sensitized mice (naïve mice) challenged with Alternaria extract (culture or cellular), but not those challenged with OVA, also showed airway eosinophilia (FIG. 10B), suggesting that prior sensitization is unnecessary for an eosinophilic response to Alternaria.

Kinetics and dose-response studies showed that airway eosinophilia was maximally induced when naïve mice were challenged with 250 μg of Alternaria culture extracts administered intranasally on days 0, 3, and 6, and when mice airway histology and cytology was examined 48 hours after the last challenge (i.e., on day 8). Dose-response experiments indicated that the magnitude of pathologic change in the Alternaria-challenge mice was dependent on the amounts of Alternaria extract used. Furthermore, at 250 μg/dose of Alternaria culture extract, about 10% of the mice died before day 8 due to massive respiratory failure.

Airway resistance was measured in mice treated with 250 μg/dose OVA, 250 μg/dose Alternaria culture or cellular extracts, 250 μg/dose of Candida culture extract, or 250 μg/mL Aspergillus culture extract in the presence of various concentrations of methacholine. Methacholine causes constriction of the airway, and can be used in patients to recreate or simulate an asthma attack. Airway resistance is measured by plethysmography, which measures the breathing rate and airway pressure of an animal.

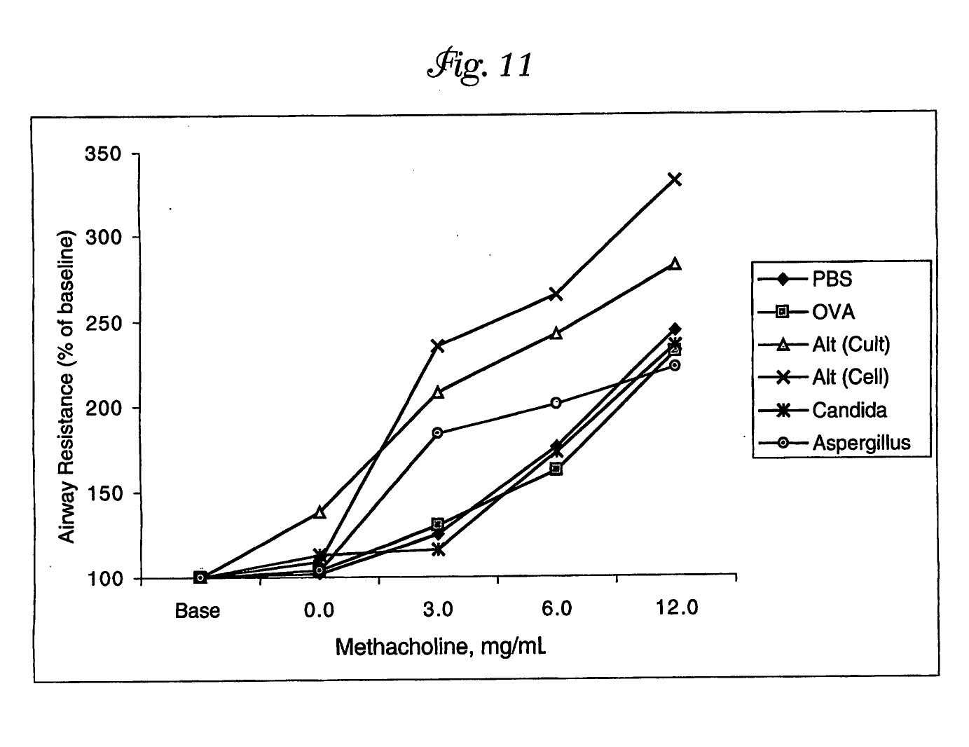

Results from these experiments demonstrated that mice exposed to Alternaria extracts (cellular or culture) experienced difficulty breathing at a much lower dose of methacholine than did control mice (FIG. 11). These findings indicate that mice exposed to Alternaria show airway hyperreactivity to methacholine, which is one of the characteristics of bronchial asthma in humans.

Pathologically, naïve mice exposed to Alternaria develop airway tissue eosinophilia, increased production of mucous by epithelial cells, and airway hyperreactivity to methacholine, suggesting that these mice show a similar phenotype as humans with bronchial asthma.

### Example 10

### Immunological Responses in Mice

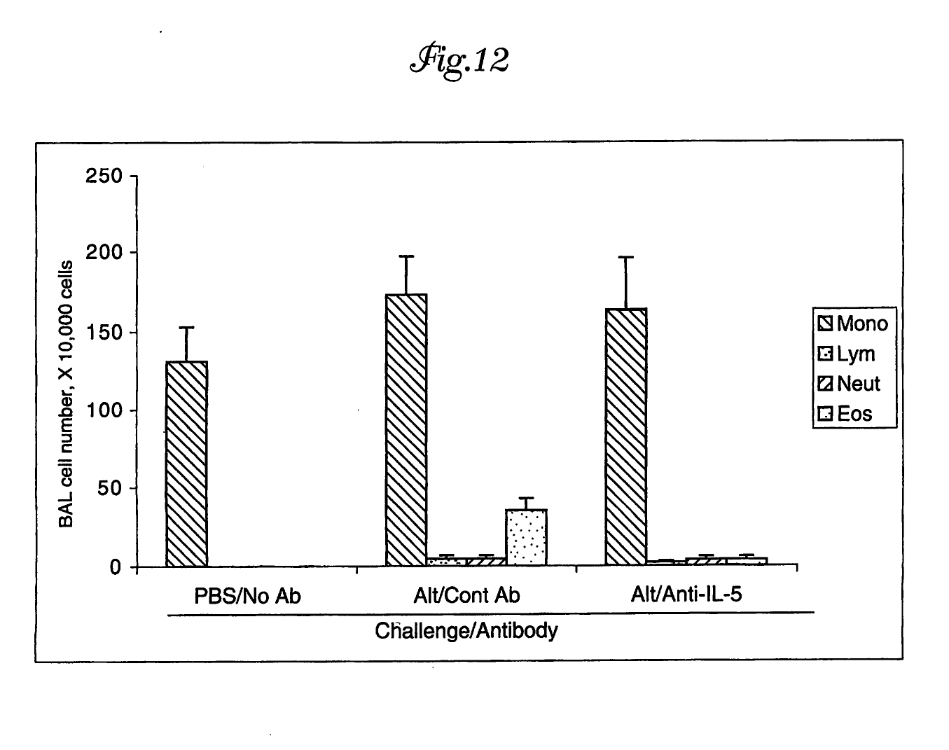

To examine the role of IL-5 in airway eosinophilia induced by Alternaria culture extracts, mice were pre-treated with 25 μg/dose of an anti-IL-5 mAb (Pharmingen) or a control antibody (rat IgG) 24 hours before being intranasally challenged at days 0, 3, and 6 with 250 μg/dose OVA, or 250 μg/dose Alternaria culture extract. The number of eosinophils was analyzed by DiffQuick staining at day 8. Alternaria-induced airway eosinophilia was inhibited by anti-IL-5 antibody (FIG. 12), suggesting that this cytokine is necessary for mice to develop airway eosinophilia.

Cytokine production in BAL fluids was examined over time following challenge with 250 μg/dose Alternaria culture extract. IL-5, IL-4, IL-13, and INF-γ were examined at 12 hour intervals following each challenge using ELISAs (Endogen, Inc.).

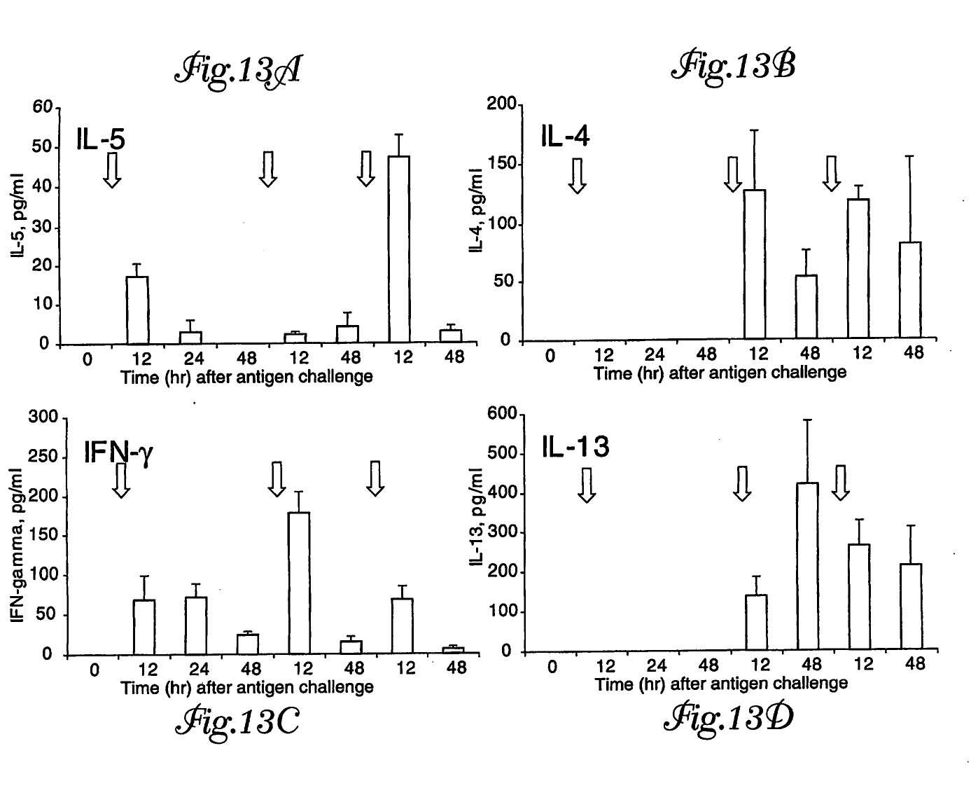

Kinetic study of IL-5 production suggests that there are two peaks of production in mouse airways exposed to Alternaria (FIG. 13A). IL-5 peaked at 12 hours after the initial Alternaria administration and peaked again at 12 hours after the third administration (FIG. 13A). IL-4 production was induced immediately following the second and third administrations of Alternaria, and levels dropped slightly by 48 hours after each administration (FIG. 13B). IFN-γ levels were elevated throughout the experiment, and reached a peak level at 12 hours following the second administration (FIG. 13C). IL-13 levels peaked 48 hours after the second administration and dropped slightly thereafter (FIG. 13D).

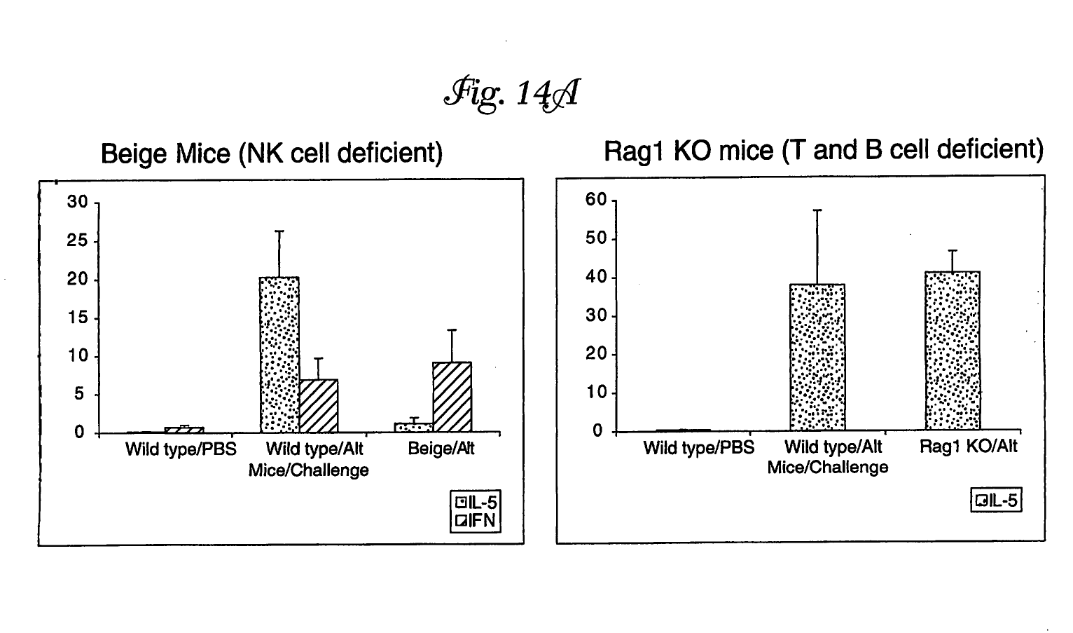

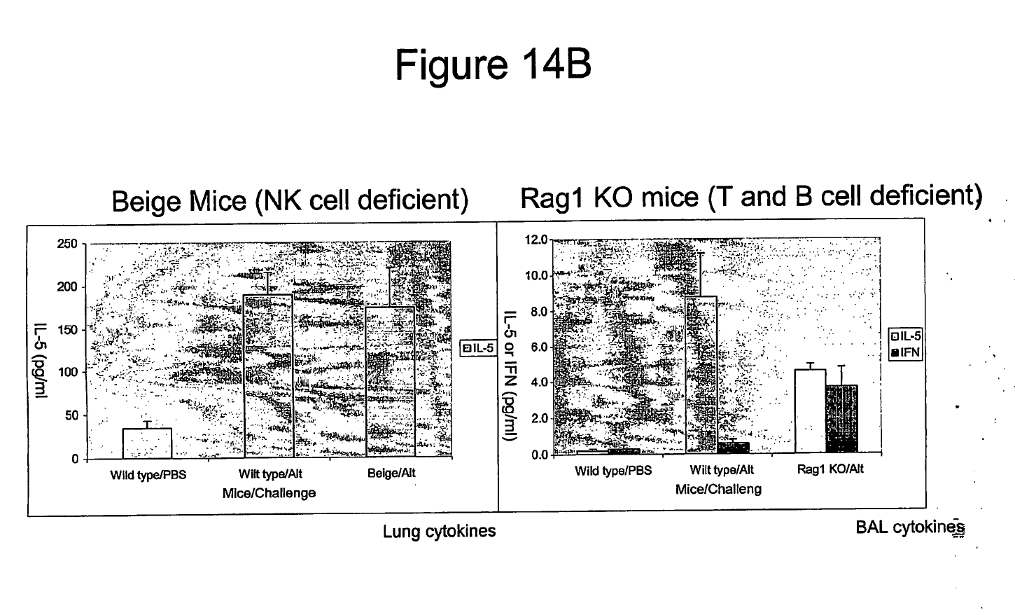

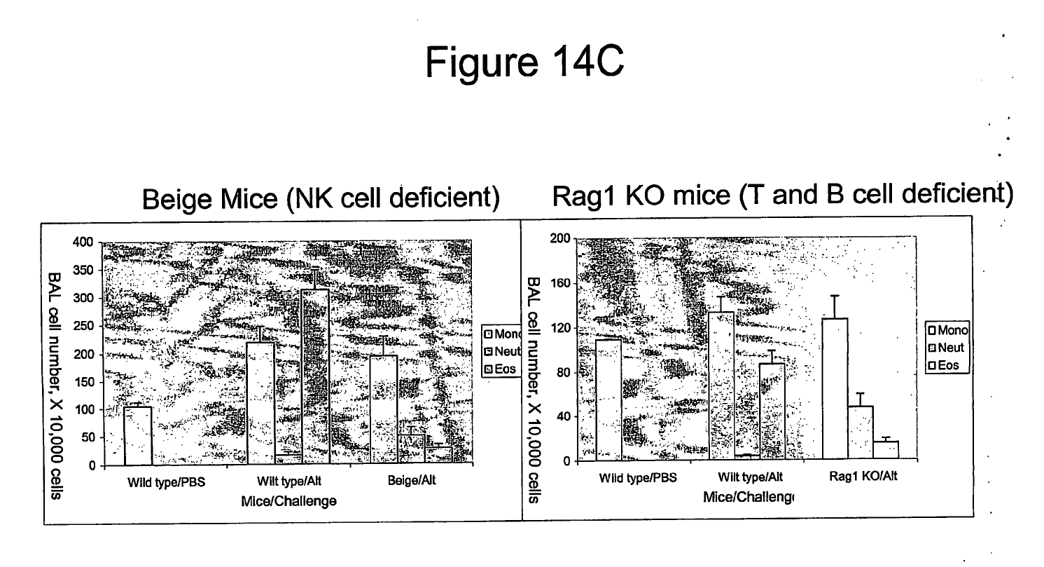

Next, experiments were performed to examine the immunological components that are activated in response to Alternaria. Beige mice, which are NK-ell deficient, were used, as were Rag1 knockout mice, which are deficient in T cells and B cells. Alternaria culture extracts were intranasally administered (250 μg/dose) to Beige or Rag1 mice. At 12 hours (FIG. 14A) and at 8 days (FIG. 14B) post-Alternaria exposure, BAL fluids were collected from the mice, and the levels of IL-5 were examined by ELISA (Endogen, Inc.). In addition, BAL fluid was collected at day 8, and the cells were examined by DiffQuick staining (FIG. 14C).

The experiments in which Rag-1 knockout mice and Beige mice were exposed to Alternaria demonstrate that very little IL-5 is produced in Beige mice at 12 hours following exposure to Alternaria (FIG. 14A, left panel), while near-wild type levels were present in Beige mice at 8 days after exposure (FIG. 14B, left panel). Results further demonstrated that IL-5 levels were near-wild type in Rag1 mice 12 hours following exposure to Alternaria (FIG. 14A, right panel), while IL-5 levels were approximately half of wild type levels at 8 days after exposure (FIG. 14B, right panel). FIG. 14C shows the immune cells that were present in BAL fluids 8 days after exposure of Beige or Rag1 mice to Alternaria. Results indicated that at 8 days following exposure, there were very few eosinophils in BAL fluids from Beige mice and Rag1 mice.

These results suggest that both T cells and NK cells are required for a mouse to develop airway eosinophilia in response to Alternaria, and further suggest that the first peak of IL-5 production (FIG. 13A) is mediated by NK cells and that the second peak (FIG. 13A) is mediated in large part by T cells. Thus, when mice are exposed to Alternaria extracts, they apparently respond initially to the antigen by innate immunity (NK cells) and subsequently develop an acquired immunity (T cells). Furthermore, IL-5 derived from both NK cells and T cells is apparently required for full development of airway eosinophilia in response to Alternaria.

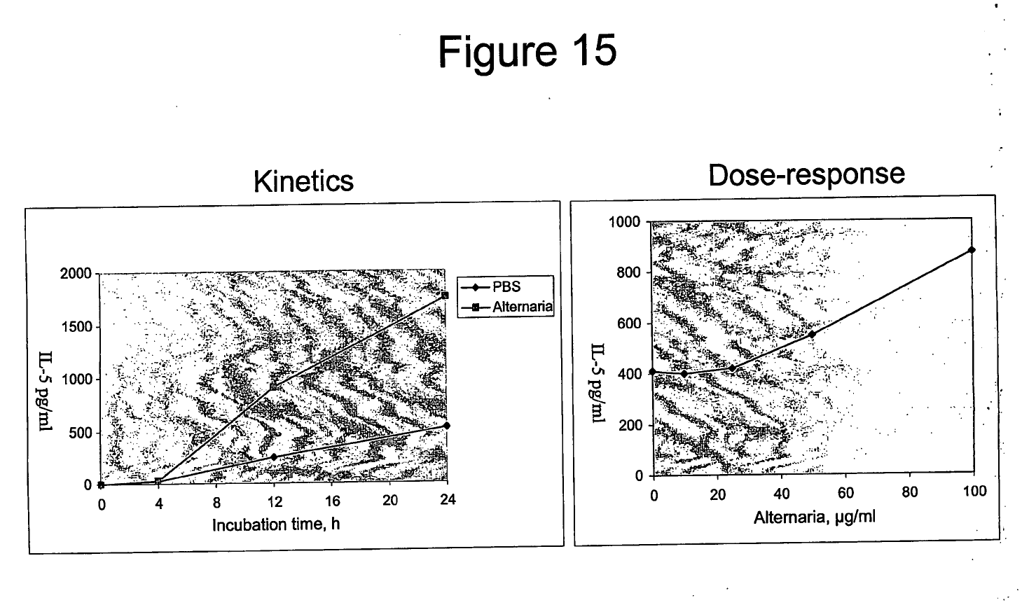

Lung tissue from three different normal mice was incubated in a petri dish at 37° C. with Alternaria culture extract. FIG. 15A shows a time course analysis of IL-5 accumulation in the presence of 50 μg/ml Alternaria in the supernatant of the petri dish. Results from these experiments indicated that IL-5 accumulated in lung tissue over the 24-hour time period examined (FIG. 15A).

FIG. 15B shows the amount of IL-5 present in the supernatant of the petri dish at 24 hours following challenge with concentrations of Alternaria culture extract ranging from 0 μg/mL to 100 μg/mL. Results from these experiments indicated that IL-5 was present in lung tissue prior to contacting it with Alternaria, and the level of IL-5 increased with increasing concentration of Alternaria (FIG. 15B).

In summary, sensitization and challenge of mice with fungal extracts induced airway eosinophilia and hyperreactivity. Surprisingly, marked airway eosinophilia was also observed in naïve (not sensitized intraperitoneally) mice challenged with Alternaria antigens. This “innate eosinophilic response” to fungi was induced by Alternaria. Other fungi (e.g., Aspergillus, or Candida) may induce an eosinophil response to a lesser degree than that induced by Alternaria. Systemic administration of anti-IL-5 antibody inhibited this eosinophilia. Specific IgE antibody to Alternaria was absent. Furthermore, airway production of IL-5 was observed as early as 4 hours after challenge of naïve mice to Alternaria. Importantly, this airway eosinophilic response was observed in T-cell deficient mice, including nude, SCID, and Rag1-knockout mice, but was strongly suppressed in mice treated with an antibody to NK cells (clone DX5). Finally, Alternaria extracts induced IL-5 production in organ culture of lungs, but not spleen, from naïve mice. Naïve mice react to Alternaria antigens with an airway IL-5 response and eosinophilia likely through an NK cell-dependent, innate immune mechanism.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of identifying an inhibitor of fungus-induced eosinophil degranulation, said method comprising determining whether or not a test compound reduces the amount of eosinophil degranulation induced by a fungal preparation, wherein said reduction indicates that said test compound is said inhibitor.

2. The method of claim 1, wherein said fungal preparation comprises a fungus extract.

3. The method of claim 2, wherein said fungus extract is selected from the group consisting of Alternaria extracts, Candida extracts, Aspergillus extracts, and Cladisporium extracts.

4. The method of claim 1, wherein said fungal preparation comprises supernatant collected from a fungus culture.

5. The method of claim 4, wherein said fungus culture is selected from the group consisting of Alternaria cultures, Candida cultures, Aspergillus cultures, and Cladisporium cultures.

6. The method of claim 1, wherein the amount of eosinophil degranulation is determined by measuring major basic protein or eosinophil-derived neurotoxin.

7. A method of identifying a fungal component that induces eosinophil degranulation, said method comprising:
(a) contacting an eosinophil with a test component, wherein said test component is a component of a fungus, and
(b) determining whether or not said test component induced said eosinophil to degranulate, wherein the presence of degranulation indicates that said test component is said fungal component.

8. The method of claim 7, wherein said test component comprises a polypeptide obtained from a fungus extract.

9. The method of claim 8, wherein said fungus extract is selected from the group consisting of Alternaria extracts, Candida extracts, Aspergillus extracts, and Cladisporium extracts.

10. The method of claim 8, wherein said polypeptide is obtained by fractionating said fungus extract.

11. The method of claim 7, wherein said test component comprises a polypeptide obtained from the supernatant of a fungus culture.

12. The method of claim 11, wherein said fungus culture is selected from the group consisting of Alternaria cultures, Candida cultures, Aspergillus cultures, and Cladisporium cultures.

13. The method of claim 11, wherein said polypeptide is obtained by fractionating said supernatant.

14. The method of claim 7, wherein the degranulation of said eosinophil is determined by measuring major basic protein or eosinophil-derived neurotoxin.

15. A method of identifying an inhibitor of eosinophil fungus attack, said method comprising determining whether or not a test compound reduces the amount of eosinophil fungus attack induced by a sample obtained from a culture comprising cells from a chronic rhinosinusitis patient and a fungal preparation, wherein said reduction indicates that said test compound is said inhibitor.

16. The method of claim 15, wherein said sample comprises a supernatant.

17. The method of claim 15, wherein said cells are peripheral blood mononuclear cells.

18. The method of claim 15, wherein said fungal preparation comprises a fungus extract.

19. The method of claim 18, wherein said fungus extract is selected from the group consisting of Alternaria extracts, Candida extracts, Aspergillus extracts, and Cladisporium extracts.

20. The method of claim 15, wherein said fungal preparation comprises media collected from a fungus culture.

21-58. (canceled)
